SEQUENCE LISTING (104 sequences)

SEQ ID NO: 1 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 2 - protein, 15 aa, Homo sapiens
EPKSCDKTHTCPPCP

SEQ ID NO: 3 - protein, 20 aa, Homo sapiens
NTKVDKTVERKCCVECPPCP

SEQ ID NO: 4 - protein, 70 aa, Homo sapiens
NTKVDKRVELKTPLGDTTHTCPRCPEPKSCDTPPPCPRCPEPKSCDTPPPCPRCPEPKSCDTPPPCPRCP

SEQ ID NO: 5 - protein, 12 aa, Homo sapiens
ESKYGPPCPSCP

SEQ ID NO: 6 - protein, 12 aa, Artificial Sequence
  Linker sequence
ESKYGPPCPPCP

SEQ ID NO: 7 - protein, 21 aa, Homo sapiens
PVPSTPPTPSPSTPPTPSPSC

SEQ ID NO: 8 - protein, 58 aa, Homo sapiens
ESPKAQASSVPTAQPQAEGSLAKATTAPATTRNTGRGGEEKKKEKEKEEQEERETKTP

SEQ ID NO: 9 - protein, 112 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 10 - protein, 99 aa, Homo sapiens
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 11 - protein, 15 aa, Homo sapiens
KHLCPSPLFPGPSKP

SEQ ID NO: 12 - protein, 48 aa, Homo sapiens
AKPTTTPAPRPPTPAPTIASQPLSLRPEACRPAAGGAVHTRGLDFACD

SEQ ID NO: 13 - protein, 45 aa, Homo sapiens
TTTPAPRPPTPAPTIASQPLSLRPEACRPAAGGAVHTRGLDFACD

SEQ ID NO: 14 - protein, 38 aa, Homo sapiens
SVVDFLPTTAQPTKKSTLKKRVCRLPRPETQKGPLCSP

SEQ ID NO: 15 - protein, 36 aa, Homo sapiens
VDFLPTTAQPTKKSTLKKRVCRLPRPETQKGPLCSP

SEQ ID NO: 16 - protein, 36 aa, Homo sapiens
APPRASALPAPPTGSALPDPQTASALPDPPAASALP

SEQ ID NO: 17 - protein, 23 aa, Homo sapiens
DSGQVLLESNIKVLPTWSTPVQP

SEQ ID NO: 18 - protein, 5 aa, Artificial Sequence
  PolyG/S hinge
GGGGS

SEQ ID NO: 19 - protein, 330 aa, Homo sapiens [sequence omitted]

SEQ ID NO: 20 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 21 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 22 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 24 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 110 aa, Homo sapiens; identical to SEQ ID NO: 23

SEQ ID NO: 26 - protein, 107 aa, Homo sapiens; identical to SEQ ID NO: 24

SEQ ID NO: 27 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 28 - protein, 106 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 29 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 30 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 33 - protein, 180 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLR

SEQ ID NO: 34 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 35 - protein, 244 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSPPIDL

SEQ ID NO: 36 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 37 - protein, 8 aa, Artificial Sequence
  CAR VH CDR1
GYSFTAYW

SEQ ID NO: 38 - protein, 8 aa, Artificial Sequence
  CAR VH CDR2
ILPGSDST

SEQ ID NO: 39 - protein, 11 aa, Artificial Sequence
  CAR VH CDR3
ARSGLYGSSQY

SEQ ID NO: 40 - protein, 10 aa, Artificial Sequence
  CAR VL CDR1
ESVDSYGNSF

SEQ ID NO: 41 - protein, 3 aa, Artificial Sequence
  CAR VL CDR2
LTS

SEQ ID NO: 42 - protein, 9 aa, Artificial Sequence
  CAR VL CDR3
QQNAEDPRT

SEQ ID NO: 43 - DNA, 24 bp, Artificial Sequence
  CAR VH CDR1 encoding DNA
ggctattcattcactgcttactgg

SEQ ID NO: 44 - DNA, 24 bp, Artificial Sequence
  CAR VH CDR2 encoding DNA
atacttccgggatccgactccact

SEQ ID NO: 45 - DNA, 33 bp, Artificial Sequence
  CAR VH CDR3 encoding DNA
gcccgcagcgggctgtacgggtcaagtcaatat

SEQ ID NO: 46 - DNA, 30 bp, Artificial Sequence
  CAR VL CDR1 encoding DNA
gagtctgtagactcctacgggaatagtttt

SEQ ID NO: 47 - DNA, 9 bp, Artificial Sequence
  CAR VL CDR2 encoding DNA
cttacttcc

SEQ ID NO: 48 - DNA, 27 bp, Artificial Sequence
  CAR VL CDR3 encoding DNA
caacagaatgctgaggacccacgaact

SEQ ID NO: 49 - protein, 111 aa, Artificial Sequence
  CAR Construct Variable Light Chain
DIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIK

SEQ ID NO: 50 - protein, 118 aa, Artificial Sequence
  CAR Construct Variable Heavy Chain
EVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSS

SEQ ID NO: 51 - DNA, 333 bp, Artificial Sequence
  CAR DNA Construct Variable Light Chain 30xx
gacatagttctcacccaatctccggcgtctctggccgtgtcacccggccaaagagctaccattacgtgtagggcgtcagagtctgtagactcctacgggaatagttttatgcactggtaccagcagaaaccgggtcagccgcccaaacttctgatctatcttacttccaatctcgagtctggcgtgcccgataggttttcaggatcaggatctggtaccgactttacactgaccattaatccagtcgaagcgaacgacgccgcaacatactattgccaacagaatgctgaggacccacgaacttttggaggagggacaaaattggagatcaag

SEQ ID NO: 52 - DNA, 354 bp, Artificial Sequence
  CAR DNA Construct Variable Heavy Chain DNA 30xx
gaggtgcaactcgttcaaagcggtgccgaggtaaagaagccaggcgagagtctcagaattagctgtaaaggctcaggctattcattcactgcttactggatcgaatgggttcgccaggcgccaggtaagggcctcgaatggattggtgagatacttccgggatccgactccactaattataacgaaaaattcaaagggcacgtgacgatctccgccgataagagtatctcaactgcgtacttgcagtggtcatcattgaaggcgagcgacacggccgtgtattattgcgcccgcagcgggctgtacgggtcaagtcaatattggggacaggggactcttgtcacggtttcaagc

SEQ ID NO: 53 - protein, 247 aa, Artificial Sequence
  CAR Construct Binding Domain CNA30xx-Variable Light-Variable Heavy
DIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKGSTSGSGKPGSGEGSTKGEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSS

SEQ ID NO: 54 - protein, 247 aa, Artificial Sequence
  CAR Construct Binding Domain CNA31xx-Variable Heavy-Variable Light
EVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSGSTSGSGKPGSGEGSTKGDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIK

SEQ ID NO: 55 - protein, 12 aa, Artificial Sequence
  CAR Construct IgG4 mutant hinge linker
ESKYGPPCPPCP

SEQ ID NO: 56 - protein, 119 aa, Artificial Sequence
  CAR Construct hinge-CH3 linker
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 229 aa, Artificial Sequence
  CAR Construct hinge-CH2-CH3 linker
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 111 aa, Artificial Sequence
  CAR Construct CD3 Zeta signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 59 - protein, 42 aa, Artificial Sequence
  CAR Construct 4-1BB Signalling Domain
KRGRKKLLYIFKQPFMRPVQTTQEEDGCSCRFPEEEEGGCEL

SEQ ID NO: 60 - protein, 460 aa, Artificial Sequence
  CAR Construct CNA3002
MSVPTQVLGLLLLWLTDARCDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKGSTSGSGKPGSGEGSTKGEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 61 - protein, 567 aa, Artificial Sequence
  CAR Construct CNA3003
MSVPTQVLGLLLLWLTDARCDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKGSTSGSGKPGSGEGSTKGEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 62 - protein, 677 aa, Artificial Sequence
  CAR Construct CNA3004
MSVPTQVLGLLLLWLTDARCDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKGSTSGSGKPGSGEGSTKGEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 63 - protein, 459 aa, Artificial Sequence
  CAR Construct CNA3102
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSGSTSGSGKPGSGEGSTKGDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 64 - protein, 566 aa, Artificial Sequence
  CAR Construct CNA3103
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSGSTSGSGKPGSGEGSTKGDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 65 - protein, 676 aa, Artificial Sequence
  CAR Construct CNA3104
MEWSWVFLFFLSVTTGVHSEVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGLYGSSQYWGQGTLVTVSSGSTSGSGKPGSGEGSTKGDIVLTQSPASLAVSPGQRATITCRASESVDSYGNSFMHWYQQKPGQPPKLLIYLTSNLESGVPDRFSGSGSGTDFTLTINPVEANDAATYYCQQNAEDPRTFGGGTKLEIKESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 66 - DNA, 1380 bp, Artificial Sequence
  CAR Construct encoding DNA CNA3002
atgtccgttcccactcaagtccttggacttttgctgctttggctgaccgacgctcgatgcgacatagttctcacccaatctccggcgtctctggccgtgtcacccggccaaagagctaccattacgtgtagggcgtcagagtctgtagactcctacgggaatagttttatgcactggtaccagcagaaaccgggtcagccgcccaaacttctgatctatcttacttccaatctcgagtctggcgtgcccgataggttttcaggatcaggatctggtaccgactttacactgaccattaatccagtcgaagcgaacgacgccgcaacatactattgccaacagaatgctgaggacccacgaacttttggaggagggacaaaattggagatcaagggtagcacgagtggttctggaaagccgggtagtggtgaggggagcacgaaaggagaggtgcaactcgttcaaagcggtgccgaggtaaagaagccaggcgagagtctcagaattagctgtaaaggctcaggctattcattcactgcttactggatcgaatgggttcgccaggcgccaggtaagggcctcgaatggattggtgagatacttccgggatccgactccactaattataacgaaaaattcaaagggcacgtgacgatctccgccgataagagtatctcaactgcgtacttgcagtggtcatcattgaaggcgagcgacacggccgtgtattattgcgcccgcagcgggctgtacgggtcaagtcaatattggggacaggggactcttgtcacggtttcaagcgaatccaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 67 - DNA, 1701 bp, Artificial Sequence
  CAR Construct encoding DNA CNA3003
atgtccgttcccactcaagtccttggacttttgctgctttggctgaccgacgctcgatgcgacatagttctcacccaatctccggcgtctctggccgtgtcacccggccaaagagctaccattacgtgtagggcgtcagagtctgtagactcctacgggaatagttttatgcactggtaccagcagaaaccgggtcagccgcccaaacttctgatctatcttacttccaatctcgagtctggcgtgcccgataggttttcaggatcaggatctggtaccgactttacactgaccattaatccagtcgaagcgaacgacgccgcaacatactattgccaacagaatgctgaggacccacgaacttttggaggagggacaaaattggagatcaagggtagcacgagtggttctggaaagccgggtagtggtgaggggagcacgaaaggagaggtgcaactcgttcaaagcggtgccgaggtaaagaagccaggcgagagtctcagaattagctgtaaaggctcaggctattcattcactgcttactggatcgaatgggttcgccaggcgccaggtaagggcctcgaatggattggtgagatacttccgggatccgactccactaattataacgaaaaattcaaagggcacgtgacgatctccgccgataagagtatctcaactgcgtacttgcagtggtcatcattgaaggcgagcgacacggccgtgtattattgcgcccgcagcgggctgtacgggtcaagtcaatattggggacaggggactcttgtcacggtttcaagcgaatccaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 68 - DNA, 2031 bp, Artificial Sequence
  CAR Construct encoding DNA CNA3004
atgtccgttcccactcaagtccttggacttttgctgctttggctgaccgacgctcgatgcgacatagttctcacccaatctccggcgtctctggccgtgtcacccggccaaagagctaccattacgtgtagggcgtcagagtctgtagactcctacgggaatagttttatgcactggtaccagcagaaaccgggtcagccgcccaaacttctgatctatcttacttccaatctcgagtctggcgtgcccgataggttttcaggatcaggatctggtaccgactttacactgaccattaatccagtcgaagcgaacgacgccgcaacatactattgccaacagaatgctgaggacccacgaacttttggaggagggacaaaattggagatcaagggtagcacgagtggttctggaaagccgggtagtggtgaggggagcacgaaaggagaggtgcaactcgttcaaagcggtgccgaggtaaagaagccaggcgagagtctcagaattagctgtaaaggctcaggctattcattcactgcttactggatcgaatgggttcgccaggcgccaggtaagggcctcgaatggattggtgagatacttccgggatccgactccactaattataacgaaaaattcaaagggcacgtgacgatctccgccgataagagtatctcaactgcgtacttgcagtggtcatcattgaaggcgagcgacacggccgtgtattattgcgcccgcagcgggctgtacgggtcaagtcaatattggggacaggggactcttgtcacggtttcaagcgaatccaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 69 - DNA, 1377 bp, Artificial Sequence
  CAR Construct encoding DNA CNA3102
atggaatggagctgggttttcctcttttttctttctgtcaccaccggggtgcattctgaagtgcagttggttcagtccggtgcggaggtaaaaaagccgggagaatcacttcggattagttgcaaaggatcagggtatagtttcaccgcgtactggattgagtgggtgagacaggcgccagggaagggccttgaatggattggtgagatccttccgggctcagattccacgaactataacgagaaatttaagggtcatgttactatctcagcagataaaagcatcagtacggcatatcttcagtggtccagtctgaaggccagtgacacggctgtgtactattgcgcccgctctggtctctacggtagttctcaatactggggccagggtacactcgtcaccgtatcttccgggtccacttcagggtccggcaagcctgggagtggtgagggatccactaagggggatatcgtactgacccaaagtccagcatctctggcagtgtcaccagggcaaagagctactattacttgtagagcttccgagtccgtggacagctatggcaactccttcatgcattggtatcaacaaaaaccaggacaaccacctaagttgctcatctacttgaccagcaacttggaatcaggggttccagaccggttctctgggtccggctccggaacggattttactcttaccataaacccggtggaggcaaacgatgctgcaacatactactgtcaacaaaacgccgaggatccccgaacattcggcggtggaacgaagttggaaattaaggagtcaaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 70 - DNA, 1698 bp, Artificial Sequence
  CAR Construct encoding DNA CNA3103
atggaatggagctgggttttcctcttttttctttctgtcaccaccggggtgcattctgaagtgcagttggttcagtccggtgcggaggtaaaaaagccgggagaatcacttcggattagttgcaaaggatcagggtatagtttcaccgcgtactggattgagtgggtgagacaggcgccagggaagggccttgaatggattggtgagatccttccgggctcagattccacgaactataacgagaaatttaagggtcatgttactatctcagcagataaaagcatcagtacggcatatcttcagtggtccagtctgaaggccagtgacacggctgtgtactattgcgcccgctctggtctctacggtagttctcaatactggggccagggtacactcgtcaccgtatcttccgggtccacttcagggtccggcaagcctgggagtggtgagggatccactaagggggatatcgtactgacccaaagtccagcatctctggcagtgtcaccagggcaaagagctactattacttgtagagcttccgagtccgtggacagctatggcaactccttcatgcattggtatcaacaaaaaccaggacaaccacctaagttgctcatctacttgaccagcaacttggaatcaggggttccagaccggttctctgggtccggctccggaacggattttactcttaccataaacccggtggaggcaaacgatgctgcaacatactactgtcaacaaaacgccgaggatccccgaacattcggcggtggaacgaagttggaaattaaggagtcaaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 71 - DNA, 2028 bp, Artificial Sequence
  CAR Construct encoding DNA CNA3104
atggaatggagctgggttttcctcttttttctttctgtcaccaccggggtgcattctgaagtgcagttggttcagtccggtgcggaggtaaaaaagccgggagaatcacttcggattagttgcaaaggatcagggtatagtttcaccgcgtactggattgagtgggtgagacaggcgccagggaagggccttgaatggattggtgagatccttccgggctcagattccacgaactataacgagaaatttaagggtcatgttactatctcagcagataaaagcatcagtacggcatatcttcagtggtccagtctgaaggccagtgacacggctgtgtactattgcgcccgctctggtctctacggtagttctcaatactggggccagggtacactcgtcaccgtatcttccgggtccacttcagggtccggcaagcctgggagtggtgagggatccactaagggggatatcgtactgacccaaagtccagcatctctggcagtgtcaccagggcaaagagctactattacttgtagagcttccgagtccgtggacagctatggcaactccttcatgcattggtatcaacaaaaaccaggacaaccacctaagttgctcatctacttgaccagcaacttggaatcaggggttccagaccggttctctgggtccggctccggaacggattttactcttaccataaacccggtggaggcaaacgatgctgcaacatactactgtcaacaaaacgccgaggatccccgaacattcggcggtggaacgaagttggaaattaaggagtcaaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 72 - protein, 28 aa, Artificial Sequence
  CAR Construct CD28 trans membrane domain
MFWVLVVVGGVLACYSLLVTVAFIIFWV

SEQ ID NO: 73 - DNA, 741 bp, Artificial Sequence
  CAR Construct ScFv CNA30xx - Variable Light-Variable Heavy DNA
gacatagttctcacccaatctccggcgtctctggccgtgtcacccggccaaagagctaccattacgtgtagggcgtcagagtctgtagactcctacgggaatagttttatgcactggtaccagcagaaaccgggtcagccgcccaaacttctgatctatcttacttccaatctcgagtctggcgtgcccgataggttttcaggatcaggatctggtaccgactttacactgaccattaatccagtcgaagcgaacgacgccgcaacatactattgccaacagaatgctgaggacccacgaacttttggaggagggacaaaattggagatcaagggtagcacgagtggttctggaaagccgggtagtggtgaggggagcacgaaaggagaggtgcaactcgttcaaagcggtgccgaggtaaagaagccaggcgagagtctcagaattagctgtaaaggctcaggctattcattcactgcttactggatcgaatgggttcgccaggcgccaggtaagggcctcgaatggattggtgagatacttccgggatccgactccactaattataacgaaaaattcaaagggcacgtgacgatctccgccgataagagtatctcaactgcgtacttgcagtggtcatcattgaaggcgagcgacacggccgtgtattattgcgcccgcagcgggctgtacgggtcaagtcaatattggggacaggggactcttgtcacggtttcaagc

SEQ ID NO: 74 - DNA, 741 bp, Artificial Sequence
  CAR Construct ScFv CNA31xx - Variable Heavy-Variable Light DNA
gaagtgcagttggttcagtccggtgcggaggtaaaaaagccgggagaatcacttcggattagttgcaaaggatcagggtatagtttcaccgcgtactggattgagtgggtgagacaggcgccagggaagggccttgaatggattggtgagatccttccgggctcagattccacgaactataacgagaaatttaagggtcatgttactatctcagcagataaaagcatcagtacggcatatcttcagtggtccagtctgaaggccagtgacacggctgtgtactattgcgcccgctctggtctctacggtagttctcaatactggggccagggtacactcgtcaccgtatcttccgggtccacttcagggtccggcaagcctgggagtggtgagggatccactaagggggatatcgtactgacccaaagtccagcatctctggcagtgtcaccagggcaaagagctactattacttgtagagcttccgagtccgtggacagctatggcaactccttcatgcattggtatcaacaaaaaccaggacaaccacctaagttgctcatctacttgaccagcaacttggaatcaggggttccagaccggttctctgggtccggctccggaacggattttactcttaccataaacccggtggaggcaaacgatgctgcaacatactactgtcaacaaaacgccgaggatccccgaacattcggcggtggaacgaagttggaaattaag

SEQ ID NO: 75 - DNA, 36 bp, Artificial Sequence
  CAR Construct CNA30xx -IgG4 mutant hinge linker DNA
gaatccaagtacggaccgccctgccccccttgccct

SEQ ID NO: 76 - DNA, 357 bp, Artificial Sequence
  CAR Construct CNA30xx -hinge-CH3 linker DNA
gaatccaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 77 - DNA, 687 bp, Artificial Sequence
  CAR Construct CNA30xx -hinge-CH2-CH3 linker DNA
gaatccaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 78 - DNA, 333 bp, Artificial Sequence
  CAR Construct CD3 Zeta signalling domain DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgccccca

SEQ ID NO: 79 - protein, 25 aa, Artificial Sequence
  CAR Construct T2A self-cleavage site
RLEGGGEGRGSLLTCGDVEENPGPR

SEQ ID NO: 80 - DNA, 333 bp, Artificial Sequence; identical to SEQ ID NO: 78
  CAR Construct T2A self-cleavage site DNA

SEQ ID NO: 81 - protein, 357 aa, Artificial Sequence
  CAR Construct EGFRt
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 82 - DNA, 1074 bp, Artificial Sequence
  CAR Construct EGFRt DNA
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 83 - protein, 107 aa, Artificial Sequence; identical to SEQ ID NO: 30
  CAR Construct IgG4 CH3 region

SEQ ID NO: 84 - protein, 110 aa, Artificial Sequence
  CAR Construct IgG4 CH2 region
APEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 85 - protein, 20 aa, Artificial Sequence
  CAR Construct light chain leader sequence
MSVPTQVLGLLLLWLTDARC

SEQ ID NO: 86 - DNA, 60 bp, Artificial Sequence
  CAR Construct light chain leader sequence DNA
atgtccgttcccactcaagtccttggacttttgctgctttggctgaccgacgctcgatgc

SEQ ID NO: 87 - protein, 19 aa, Artificial Sequence
  CAR Construct heavy chain leader sequence
MEWSWVFLFFLSVTTGVHS

SEQ ID NO: 88 - DNA, 57 bp, Artificial Sequence
  CAR Constructs heavy chain leader sequence DNA
atggaatggagctgggttttcctcttttttctttctgtcaccaccggggtgcattct

SEQ ID NO: 89 - DNA, 36 bp, Artificial Sequence
  CNA31xx -IgG4 mutant hinge linker DNA
gagtcaaagtacggaccgccctgccccccttgccct

SEQ ID NO: 90 - DNA, 357 bp, Artificial Sequence
  CAR Construct CNA31xx -hinge-CH3 linker DNA
gagtcaaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 91 - DNA, 687 bp, Artificial Sequence
  CAR Construct CNA31xx -hinge-CH2-CH3 linker DNA
gagtcaaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 92 - DNA, 84 bp, Artificial Sequence
  CAR Construct xx02/03 - CD28 trans membrane domain DNA
atgttctgggtgctggtggtggtcggaggcgtgctggcctgctacagcctgctggtcaccgtggccttcatcatcttttgggtg

SEQ ID NO: 93 - DNA, 84 bp, Artificial Sequence
  CAR Construct xx04 - CD28 trans membrane domain DNA
atgttctgggtgctggtggtggtgggcggggtgctggcctgctacagcctgctggtgacagtggccttcatcatcttttgggtg

SEQ ID NO: 94 - DNA, 126 bp, Artificial Sequence
  CAR Construct 4-1BB Signalling Domain DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 95 - protein, 8 aa, Artificial Sequence
  Antibody 18G7.1 heavy chain CDR1
GYTFSGYW

SEQ ID NO: 96 - protein, 11 aa, Artificial Sequence
  Antibody 18G7H6A1 heavy chain CDR3
ARSGYYGSSQY

SEQ ID NO: 97 - protein, 9 aa, Artificial Sequence
  Antibody 18G7.1 light chain CDR3
QQNNEDPRT

SEQ ID NO: 98 - protein, 18 aa, Artificial Sequence
  Scfv fusion domain
GSTSGSGKPGSGEGSTKG

SEQ ID NO: 99 - protein, 118 aa, Artificial Sequence
  Antibody Fragment Ab 18G7.1 heavy chain
QVQLQQSGAELVKPGASVKISCKATGYTFSGYWIEWVKQRPGHGLEWIGEILPGSDSTNYNEKFKGKATFTADTSSNTVYMQFSSLTSEDSAVYYCARSGYYGSSQYWGQGTTLTVSS

SEQ ID NO: 100 - protein, 111 aa, Artificial Sequence
  Antibody Fragment Ab 18G7.1 light chain
NIVLTQSPASLAVSLGQRATISCRASESVDSYGNSFMHWYQQKPGQPPKLLIYLTSNLESGVPARFSGSGSRTDFTLTIDPVEADDAATYYCQQNNEDPRTFGGGTKLEIK

SEQ ID NO: 101 - protein, 124 aa, Artificial Sequence
  Antibody Fragment 18G7H6A1 heavy chain
EVQLVQSGAEVKKPGESLRISCKGSGYSFTAYWIEWVRQAPGKGLEWIGEILPGSDSTNYNEKFKGHVTISADKSISTAYLQWSSLKASDTAVYYCARSGYYGSSQYWGQGTLVTVSSASTKGP

SEQ ID NO: 102 - protein, 111 aa, Artificial Sequence; identical to SEQ ID NO: 49
  Antibody Fragment 18G7H6A1 light chain

SEQ ID NO: 103 - DNA, 333 bp, Artificial Sequence
  CAR Construct Variable Heavy Chain 31xx DNA
gatatcgtactgacccaaagtccagcatctctggcagtgtcaccagggcaaagagctactattacttgtagagcttccgagtccgtggacagctatggcaactccttcatgcattggtatcaacaaaaaccaggacaaccacctaagttgctcatctacttgaccagcaacttggaatcaggggttccagaccggttctctgggtccggctccggaacggattttactcttaccataaacccggtggaggcaaacgatgctgcaacatactactgtcaacaaaacgccgaggatccccgaacattcggcggtggaacgaagttggaaattaag

SEQ ID NO: 104 - DNA, 354 bp, Artificial Sequence
  CAR Construct Variable Heavy Chain 31xx DNA
gaagtgcagttggttcagtccggtgcggaggtaaaaaagccgggagaatcacttcggattagttgcaaaggatcagggtatagtttcaccgcgtactggattgagtgggtgagacaggcgccagggaagggccttgaatggattggtgagatccttccgggctcagattccacgaactataacgagaaatttaagggtcatgttactatctcagcagataaaagcatcagtacggcatatcttcagtggtccagtctgaaggccagtgacacggctgtgtactattgcgcccgctctggtctctacggtagttctcaatactggggccagggtacactcgtcaccgtatcttcc

(19) United States
(12) Patent Application Publication
(10) Pub. No.: US 2024/0033356 A1
(43) Pub. Date: Feb. 1, 2024

(54) CHIMERIC ANTIGEN RECEPTOR T CELL AND METHOD

(71) Applicant: CARINA BIOTECH PTY LTD, Mawson Lakes, South Australia (AU)

(72) Inventors: Justin Taylor COOMBS, Wattle Park, South Australia (AU); Shaun Reuss MCCOLL, Myrtle Bank, South Australia (AU); Simon Charles BARRY, Longwood, South Australia (AU)

(21) Appl. No.: 18/037,676

(22) PCT Filed: Nov. 18, 2021
(86) PCT No.: PCT/AU2021/051374

§ 371 Date: May 18, 2023

(30) Foreign Application Priority Data
Nov. 18, 2020 (AU) 2020904256

(51) Int. Cl. A61K 39/00; A61K 35/17; A61P 35/00

(52) U.S. Cl. CPC A61K 39/4631; A61K 35/17; A61K 39/464402; A61P 35/00; A61K 2239/21; A61K 2239/13

(57) ABSTRACT

N(57) Abstract: There is a need for new and alternative options for immune therapy of proliferative diseases such as cancer. This need C\11 may be address by providing a chimeric antigen receptor having an extracellular domain including a binding domain which recognises Nthe cancer-stem-cell-associated protein Lgr5; a transmembrane domain; and an intracellular signalling domain that activates a cellular .,, function. Such CAR T cells can be used to kill cancer cells and treat or prevent cancer in a subject.

The present application claims priority from the Australian provisional application 2020904256, the entire contents of which is incorporated herein.

Technical Field

The present invention relates to chimeric antigen T cell receptors, immune cells expressing chimeric antigen T cell receptors, pharmaceutical compositions comprising chimeric antigen T cell receptors, methods of using chimeric antigen T cell receptors and T cells bearing such T cell receptors for use in the prevention and/or treatment of proliferative diseases such as cancer.

Background of Invention

The following discussion of the background to the invention is intended to facilitate an understanding of the invention. However, it should be appreciated that the discussion is not an acknowledgement or admission that any of the material referred to was published, known or part of the common general knowledge as at the priority date of any one of the claims of this specification.

The immune system actively maintains the integrity of the body of a subject by preventing infection by a range of opportunistic microorganism and preventing aberrant cell growth such a neoplastic, or pre-neoplastic, cells. Due to the specificity and efficacy of the immune system, there have been many attempts to manipulate the adaptive immune system to specifically target neoplastic cells. These techniques are collectively referred to as immuno-oncology.

Immuno-oncology comprises many different techniques including the use of antibodies, cancer antigen vaccination, cytokine treatment and adoptive T cell transfer.

Perhaps the most utilised form of immuno-oncology is the development of specific monoclonal antibodies directed against tumour-associated antigens. Antibodies demonstrate a range of features that make them excellent options for immuno- oncology. Within one individual (human) it is estimated that as many as 10 11 to 10 12 unique antibodies can be generated each with a different antigen specificity and affinity. As such, antibodies can be generated to almost any surface antigen. Further, antibodies can be readily recombinantly produced in vitro using immortalised hybridoma cell lines. This means that they can be produced in large quantities.

Moreover, antibodies can be formulated (for example lyophilised) to be stable over prolonged periods of time thereby allowing for easy storage and distribution. Finally, the dosage of antibodies can be easily controlled to increase responsiveness, or to reduce side-effects should they develop.

Antibodies can be utilised to direct targeted cell killing via antibody-

dependent cellular cytotoxicity (ADCC) whereby binding of an antibody to a target directs lysis of the antibody bound cell. Alternatively, antibodies can be used to inhibit the functionality of receptors and signalling molecules which are expressed on cancer cells.

As a result of the above properties, antibodies have become increasingly

popular in immuno-oncology. However, they do have some limitations. For example, the size of antibodies reduces their ability to access cells within a solid tumour which are often poorly vascularised. Further, cancer cells will routinely adapt such that they express low levels, or none, of the antigens being targeted. As such, antibodies typically prolong survival in cancer infected individuals, rather than cure patients.

Recently, scientists and physicians have begun to explore the use of the cellular arm of the adaptive immune system to treat cancers. For example, T-cells, known as tumour-infiltrating leukocytes (TILs), have been isolated from excised tumours. These TILS can be selectively expanded based on responsiveness to individual tumour-associated antigens and administered back to a patient. In some instances, when administered with additional treatments, these TILs have resulted in the patient undergoing complete remission (NIH (2018) “New approach to immunotherapy leads to complete response in breast cancer patient unresponsive to other treatments”). The success of cellular treatments, when other frontline treatments have failed, may be attributable to the ability of T cells to actively infiltrate tumours and to induce and coordinate the response of other parts of the immune system.

However, the success rate of these cellular treatments are extremely variable and the treatments largely rely on characterisation of the unique antigenic properties of individual tumours, and the identification of a small number of TILs reactive with these unique antigens. This results in extremely high costs, long timeframes for generation of the TILs, and in some cases no appropriate TILs can be isolated and expanded.

Consequently, there is a need for new and alternative options for immune therapy of proliferative diseases such as cancer.

Summary of Invention

The present invention is predicated, in part, on the development of chimeric-

antigen receptor (CAR) T cells that recognise Lgr5 — a tumour associated antigen.

Leucine-rich repeat-containing G-protein coupled receptor 5 (Lgr5) is a receptor, which when bound by its cognate ligand, activates Wnt signalling. Lgr5 has been identified as a marker for cancer stem cells with elevated signalling being implicated in cancer development and recurrence.

Accordingly, the present invention provides a chimeric antigen receptor including, or consisting of: an extracellular domain including a binding domain which recognises Lgr5; a transmembrane domain; and an intracellular signalling domain that activates a cellular function.

Chimeric antigen receptors (CARs) are artificially constructed proteins that

upon expression on the surface of a cell can induce an antigen-specific cellular response. A CAR utilises intracellular portions that, when activated, cause a signalling cascade within the host cell. Chimeric antigen receptors further include an extracellular domain with a binding domain that determines the antigen specificity of the CAR. As such, CARs can be designed to mimic the function of an antigen receptor, such as a T- cell receptor, but with customisable antigen specificity and customisable intracellular signalling. However, unlike T-cell receptors, CARs can be designed to be activated independently of major histocompatibility (MHC) molecules such as human leukocyte antigen (HLA) and, depending on the design of the intracellular portion, independent of additional or extrinsic co-stimulation.

Therefore, when expressed in an appropriate host cell such as a T cell, the CAR of the present invention can induce cellular activity which can result in the killing of a cell expressing Lgr5. As such, the CAR of the present invention can be used to kill, or induce the killing of, cells expressing Lgr5.

Lgr5 includes a solenoid protein domain comprising 17 leucine rich repeats

which form a horseshoe shape having both a convex surface, and a concave ligand- binding surface. In some embodiments, the binding domain of the CAR recognises an epitope on the convex surface of Lgr5. In some embodiments, the epitope is within leucine rich repeats 6 to 9 of Lgr5.

Antibodies have been generated which include biding portions (such as

CDRs) that bind to Lgr5. These include 18G7H6A3 (BNC101), 18G7H6A1 and 18G7.1. Therefore, in some embodiment, the binding domain includes at least a binding portion of an antibody selected from 18G7H6A3, 18G7H6A1 or 18G7.1.

As would be understood in the art, antibodies include variable regions defined by a variable heavy (VH) chain and a variable light (VL) chain. Each of these variable regions include four framework regions interspace by three complementarity- determining regions (CDRs) resulting in a total of six CDRs. However, single chains of antibodies can specifically bind to epitopes. Primarily variable heavy chains can be used to form single domain antibodies (sdAbs). Further, single-chain variable fragment (scFv) fusion proteins can be generated whereby variable heavy and light chains are fused together via a fusion peptide (fusion-linker). Alternatively, binding fragments of antibodies known as Fab (fragment antigen biding) can be generated by enzymatic digestion of an antibody to separate the Fc fragment from the Fab fragment. The digestion can be at the hinge region creating two Fabs from an antibody, or below the hinge to create an F(ab)2 which comprises the two Fab regions of the antibody linked by the antibody disulfide bridges.

Accordingly, in some embodiments, the binding domain of the CAR of the present invention is a single-domain antibody including a sequence identical to a VH or VL chain of an antibody that binds to Lgr5, or a sequence identical to a Fab fragment of an antibody that binds to Lgr5, or a sequence identical to a single-chain variable fragment (scFv) comprising the VH and VL regions of an antibody that binds to Lgr5.

In some embodiments, the binding domain of the CAR includes at least the variable heavy chain of an antibody that binds to Lgr5. However, variable light chains in the form of single domain antibodies can also specifically bind to antigens. Accordingly, in some embodiments, the binding domain includes at least the variable 5 light chain of an antibody that binds to Lgr5.

As disclosed, each variable chain includes three CDRs. Therefore, in some embodiments the binding domain of the CAR includes: a heavy chain CDR1 having an amino acid sequence as set forth in SEQ ID No. 37 or having an amino acid sequence as set forth in SEQ ID No. 37 with 1, 2 or 3 amino acid modifications; and a heavy chain CDR2 having an amino acid sequence as set forth in SEQ ID No. 38, or having an amino acid sequence as set forth in SEQ ID No. 38 with 1, 2 or 3 amino acid modifications; and a heavy chain CDR3 having an amino acid sequence as set forth in SEQ ID No. 39, or having an amino acid sequence as set forth in SEQ ID No. 39 with 1, 2 or 3 amino acid modifications. In some embodiments, the binding domain includes 15 amino acid sequences identical to SEQ ID No. 37, SEQ ID No. 38 and SEQ ID No. 39.

In some embodiments, the binding domain of the CAR includes: a light chain CDR1 having an amino acid sequence set forth in SEQ ID No. 40 or having an amino acid sequence as set forth in SEQ ID No. 40 with 1, 2 or 3 amino acid modifications; and a light chain CDR2 having an amino acid sequence set forth in SEQ ID No. 41, or having an amino acid sequence as set forth in SEQ ID No. 41 with 1, 2 or 3 amino acid modifications; and a light chain CDR3 having an amino acid sequence set forth in SEQ ID No. 42 or having an amino acid sequence as set forth in SEQ ID No. 42 with 1, 2 or 3 amino acid modifications.

In some of the embodiments, wherein the binding domain is a single-chain 25 variable fragment, the C-terminus of a variable heavy (VH) chain is linked to the N- terminus of a variable light (VL) chain. In some alternative embodiments, the single- chain variable fragment includes the C-terminus of the VL chain linked to the N- terminus of the VH chain. In a preferred form of these embodiments, the VH chain includes; a CDR1 having the sequence of SEQ ID No. 37, a CDR2 having the sequence 30 of SEQ ID No. 38, and a CDR3 having the sequence of SEQ ID No. 39, and the VL chain includes; a CDR1 having the sequence of SEQ ID No. 40, a CDR2 having the sequence of SEQ ID No. 41, and a CDR3 having the sequence of SEQ ID No. 42, and wherein each CDR can have 1, 2 or 3 amino acid modifications.

In some embodiments, the binding domain includes a heavy chain CDR1 having SEQ ID No. 95, and any of the heavy chain CDR2 or CDR3 set forth in SEQ ID Nos. 38, 39 or 96. In some embodiments, the binding domain includes a heavy chain

CDR3 having SEQ ID No. 96, and any of the heavy chain CDR1 or CDR2 set forth in SEQ ID Nos. 37, 95 or 38.

In some embodiments, the binding domain of the CAR includes SEQ ID No. 49 and/or SEQ ID No. 50, or variants thereof having at least 80% sequence identity to SEQ ID No. 49 or 50. In some embodiments, the sequence of the binding domain has at least 90% sequence identity to SEQ ID No. 49 or 50. In some embodiments, the sequence of the binding domain has at least 95% sequence identity to SEQ ID No. 49 or 50.

In some embodiments, the VL and VH are linked by a fusion domain which includes, or consist of, the sequence of SEQ ID No. 98. Accordingly, in some embodiments, the binding domain includes SEQ ID No. 53 or SEQ ID No. 54, or variants thereof having at least 80% sequence identity to SEQ ID No. 53 or 54. In some embodiments, the sequence of the binding domain has at least 90% sequence identity to SEQ ID No. 53 or 54.

The antigen recognition domain of a CAR can be directly connected to the

transmembrane domain. However, it may be advantageous to provide a linker domain between the antigen recognition domain and the transmembrane domain. CAR T cells have been designed and produced that function without the inclusion of a linker domain, and therefore, in this context, a linker domain may not be essential to the function of CARs.

However, some CARs fail to function in the absence of a linker domain or may not function optimally. Therefore, in some embodiments of the CAR, the extracellular domain includes a linker domain which links the binding domain to the transmembrane domain.

The length of the linker domain may alter the functionality of the CAR and T cells expressing the CAR. In some embodiments of the CAR, the linker domain is at least 12 amino acids in length. In some embodiments, the linker domain is, or is at least, about 12 amino acids in length. In some embodiments, the linker domain is, or is at least, 119 amino acids in length. In some embodiments, the linker domain is, or is at least, about 119 amino acids in length. In some embodiments, the linker domain is, or is at least, 229 amino acids in length. In some embodiments, the linker domain is, or is at least, about 229 amino acids in length. In some embodiments, the linker domain is from 12 amino acids long to 229 amino acids long. In some embodiments, the linker domain is from about 12 amino acids long to about 229 amino acids long. In some embodiments, the linker domain is from 119 amino acids long to 229 amino acids long. In some embodiments, the linker domain is from about 119 amino acids long to about 229 amino acids long.

A range of peptide sequences can be utilised to provide the linker domain of the CAR. In some embodiments of the present invention, the linker domain includes an amino acid sequence identical to the sequence of an immunoglobulin hinge such as the IgG4 hinge. In some embodiments, the linker region includes a sequence identical to a modified version of the IgG4 hinge. In some embodiments, the modified version of the IgG4 hinge includes 1, 2 or 3 amino acid modifications, preferably 1 amino acid modification.

In some embodiments, the linker domain includes an amino acid sequence identical to the sequence of a CH3 region of an immunoglobulin, such as the CH3 region of IgG4. In some embodiments, the linker domain includes an amino acid sequence identical to the sequence of a CH2 region of an immunoglobulin, such as the CH2 region of IgG4. In some embodiments, the linker domain includes a combination of the hinge region, the CH3 region and/or the CH2 region.

In some embodiments, the linker domain includes, or consists of, a sequence selected from SEQ ID No. 55, SEQ ID No. 56 or SEQ ID No. 57, or functional variants thereof.

Once a T cell expressing a CAR recognises its cognate antigen, the

intracellular signalling domain of the CAR signals activation of the T cell. As such, the intracellular signal of the CAR can influence the type and magnitude of cellular activation.

In some embodiments, the intracellular signalling domain includes a portion having an amino acid sequence identical to a signalling portion of an activation receptor. In some embodiments, the activation receptor is a member of the CD3 co- receptor complex or an Fc receptor. Particularly envisaged signalling domains include an amino acid sequence identical to at least a signalling portion of CD3- (CD3 zeta), or a signalling portion of an FccRl or FcyRl.

In some embodiments, the intracellular signalling domain of the CAR includes a portion having an amino acid sequence identical to a signalling portion of a co-stimulatory receptor. In some embodiments, the co-stimulatory receptor is selected from the group consisting of CD27, CD28, CD30, CD40, DAP10, OX40, 4-1BB (CD137) and ICOS. In some embodiments, the co-stimulatory receptor is 4-1BB.

In some embodiments, the intracellular domain includes an amino acid sequence identical to at least a signalling portion of CD3- (CD3 zeta) and an amino acid sequence identical to a signalling portion of 4-1BB, or functional variants thereof.

In some embodiments, the intracellular domain includes an amino acid sequence identical to SEQ ID No. 58 and/or SEQ ID No. 59, or functional variants thereof.

In some embodiments, the CAR includes, or consists of, an amino acid

sequence selected from the group consisting of: SEQ ID No. 60, 61, 62, 63, 64 or 65, or functional variants thereof. In some preferred embodiments, the CAR includes, or consists of, an amino acid sequence selected from the group consisting of: SEQ ID No. 62, 63, 64 or 65, or functional variants thereof.

Further provided is a nucleic acid molecule encoding a CAR as disclosed

above or nucleic acid construct including the nucleic acid molecule. In some embodiments, the nucleic acid construct is a vector. In some embodiments, the expression of the nucleic acid molecule in the nucleic acid construct is under the control of a transcriptional control sequence. In some embodiments, the transcriptional control sequence is a constitutive promoter. In some embodiments, the promoter is selected from the group consisting of: simian virus 40 (SV40), cytomegalovirus (CMV), P-actin, Ubiquitin C (UBC), elongation factor-1 alpha (EF1A), phosphoglycerate kinase (PGK) and CMV early enhancer/chicken R actin (CAGG). In some embodiments the promoter is a CMV promoter.

The nucleic acid, or nucleic acid construct, of the present invention can be

for use in preparing a genetically modified cell or for use in preparing a viral vector or for use in preparing a medicament, or used in methods of genetically modifying a cell, or preparing a viral vector of a medicament.

Accordingly, also provided herein is a viral vector including a nucleic acid molecule, or a nucleic acid construct, encoding for and/or expressing a CAR as described herein. In some embodiments, the viral vector is a lentivirus.

In some embodiments, the viral vector is used in a method of preparing a genetically modified cell. In some embodiments, the viral vector is for use in the preparation of a medicament for the treatment of cancer or for the killing of a cell expressing or aberrantly-expressing Lgr5.

Further provided by the present invention is a cell or a genetically modified cell including: the chimeric antigen receptor as described herein, or a nucleic acid molecule or construct as described here, or a genomically integrated form of the nucleic acid molecule or construct.

In some embodiments, the genetically modified cell is a leukocyte, a

Peripheral Blood Mononuclear Cell (PBMC), a lymphocyte, a T cell, a natural killer cell or a natural killer T cell.

In some embodiments the T cell is a CD4+T cell, or a CD8+T cell.

The present disclosure also provides, a method of killing a target cell expressing or aberrantly-expressing Lgr5, the method including exposing the target cell to a cell or genetically modified cell expressing a CAR, wherein the CAR targets Lgr5. In some embodiments, the CAR includes any one or more of the features described herein.

In some embodiments, the cell or genetically modified cell is autologous to the target cell. In some embodiments, the cell or genetically modified cell is allogeneic to the target cell. In some embodiments, the target cell is within the body of a subject. In some embodiments, the target cell is a cancer cell.

5 In some embodiments of the method of killing a target cell, the method

further includes analysing the surface expression of Lgr5 on the target cells prior to exposing the target cell to a cell or genetically modified cell expressing a chimeric antigen receptor. In some embodiments, wherein the target cell is a cancer cell, the surface expression of Lgr5 on the cancer cells is compared to comparable non- 10 cancerous cells to identify aberrant expression.

The present disclosure also provides, a method of preventing or treating a patient having cancer, the method including exposing the patient to a CAR, wherein the CAR targets Lgr5. Preferably, the CAR is expressed in a cell or a genetically modified cell such as those described herein. Accordingly, in some embodiments, the method of 15 preventing or treating a patient having cancer includes exposing the patient to a cell including or expressing a CAR as described herein. In some embodiments, the cell is autologous to the patient. In some embodiments, the cell is allogeneic to the patient. In some embodiments, the method of preventing or treating cancer in a patient further includes identifying the presence of cancer stem cells in the patient.

20 In some embodiments of the methods for killing a cancer cell or preventing

or treating cancer is a patient, the cancer cell is selected from one or more of; breast, pancreatic, prostate, colon, colorectal, gastrointestinal lung (NSCLC), lymphoma, ovarian or B-cell lymphoma. In some embodiments, the cancer cell is selected from one or more of; colorectal, B-cell lymphoma or ovarian cancer. In some embodiments 25 the cancer is colon, colorectal or another gastrointestinal cancer. In some embodiments, the cancer is metastatic or is recurrent cancer.

Further provided is a pharmaceutical composition including a CAR, nucleic acid, a vector or a genetically modified cell as described herein and a pharmaceutically acceptable carrier, excipient or diluent. In some embodiments, the pharmaceutical 30 composition includes a cytokine.

### BRIEF DESCRIPTION OF DRAWINGS

Notwithstanding any other forms which may fall within the scope of the present invention, preferred embodiments of the invention will now be described, by way of example only, with reference to the accompanying drawings in which:

FIG. 1 is an alignment of the CDRs of the heavy and light chains of the

antibodies 18G7.1, 18G7H6A1 and embodiments of the chimeric antigen receptors exemplified herein.

FIG. 2 is an alignment of the variable heavy and light chain regions of the antibodies 18G7.1 and 18G7H6A1 and the variable heavy and light chain of embodiments of the chimeric antigen receptors exemplified herein. The CDRs are indicated by the boxes.

FIG. 3 is a schematic illustration of a chimeric antigen receptor (CAR) according to embodiments of the present invention illustrating three different linker domains of 12(4a), 119(4b) and 229(4c) amino acids in length.

FIGS. 4A and 4B are schematic illustration showing two embodiments of

the binding domain of a CAR in accordance with the present invention. Specifically illustrated are two scFv fusion proteins directed against Lgr5 and including antibody variable heavy (VH) and variable light (VL) chains linked by a fusion domain.

FIGS. 5A and 5B are flow cytometry histograms of lymphocytes (based on forward-scatter and side-scatter gating) and stained for CD4+, CD8+and EGFR expression (as a proxy marker for CAR expression), FIG. 5A illustrates CAR expression 5 days after transduction with CNA3002, CNA3003 and CNA3004, as well as and untransduced T cell. FIG. 5B illustrates CAR expression 5 days after transduction with CNA3102, CNA3103 and CNA3104 as well as untransduced T cells.

FIG. 6A and 6B are flow cytometry histograms of lymphocytes (based on

forward-scatter and side-scatter gating) and stained for CD4+, CD8+and EGFR expression (as a marker for CAR expression). FIGS. 6A illustrates CAR expression days after transduction with CNA3002, CNA3003 and CNA3004 and untransduced T cell. FIG. 6B illustrates CAR expression 15 days after transduction with CNA3102, CNA3103 and CNA3104 and untransduced T cells.

FIG. 7 is a flow cytometry histogram of Lgr5 expression in wild type CHO cells and CHO cells over-expressing Lgr5, which were either unstained and stained with the anti-Lgr5 antibody BNC101.

FIGS. 8A and 8B illustrate the efficacy of CAR transduced T cells in lysing Lgr5 expressing targets. Specifically, FIG. 8A illustrates cytotoxicity (killing) assays demonstrating that T cells transduced with one of CNA3002, CNA3004, CNA3102, CNA3103 and CNA3104 can effectively lyse CHO cells over-expressing Lgr5 at target cell to effector cell ratios of 10:1, 3:1 and 1:1, while untransduced T cells only minimally lyse the same target cells. FIG. 8B illustrates that the same transduced T cells only effect minimal killing to CHO cells which do not over-express Lgr5.

FIGS. 9A to 9G illustrate the efficacy of CNA3002, CNA3003, CNA3004, CNA3102, CNA3103 and CNA3104 in killing a range of cancer cell lines. Cancer cell lines assayed include: (9A) LoVo cells (metastatic colon epithelial cell cancer); (9B) LIM1215 cells (colorectal epithelial cell carcinoma); (9C) Raji cells (Burkitt's lymphoma); (9D) Namalwa cells (Burkitt's lymphoma); (9E) OVCAR3 (ovarian carcinoma); (9F) SHY-5Y-SY (neuroblastoma); and (9G) BE(2)-M17 (neuroblastoma). These assays were performed with effector CAR T cell to target cancer cells (E:T) ratios of 10:1, 3:1 and 1:1

FIGS. 10A and 10B illustrate the outcome of repeats of the killing assays shown in FIGS. 9A and 9B but with effector to target cell (E:T) ratios of 10:1 to 1:30.

FIG. 11 illustrates the efficacy of CNA3002, CNA3004, CNA3102, CNA3103 and CNA3104 in killing primary ovarian cancer cells at effector to target cell (E:T) ratios of 10:1 and 1:1.

FIGS. 12A to 12C illustrate the effect of Lgr5 blocking antibodies in CAR T cell mediated killing of LoVo target cells. The figures specifically illustrate the efficacy of CNA3002, CNA3004, CNA3102, CNA3103 and CNA3104 in killing primary ovarian cancer cells at effector to target cell (E:T) ratios of 10:1 and 1:30.

FIGS. 13A illustrate the in vivo efficacy (as measured by tumour growth) of CNA3004, CNA3102, CNA3103 and CNA3104 in a murine xenograft model of cancer.

FIGS. 14A and 14B illustrate the survival time (14A) and days tumour free (14B) in a murine xenograft model of cancer.

### DETAILED DESCRIPTION

Leucine-rich repeat-containing G-protein coupled receptor 5 (Lgr5) is a seven-transmembrane protein of the class A Rhodopsin-like family of GPCRs. Lgr5, when bound by its ligand R-Spondin, interacts with Wnt receptors Frizzled and LPR5/6 to potentiate Wnt/p-catenin signalling (Carmon, K. S. et al., PNAS., 2011; 108, 11452- 7).

When Lgr5 is activated, p-catenin accumulates in the cytosol of the cell and translocates to the nucleus where it activates proto-oncogenic genes including c-myc, cyclinD1 and survivin. Under normal conditions Lgr5 is essential for embryonic development as well as cellular plasticity, proliferation and differentiation of several lineages of adult stem cells. As such, Lgr5 is a marker for adult stem cells in several tissue including, mammary tissues, the small and large intestines and hair follicles (Xu, L et al., Stem Cell Res Ther., 2019; 10:219).

Notably, Lgr5 has been identified as being upregulated in a various cancer types including adenocarcinoma, basal cell carcinomas, glioblastoma, hepatocellular carcinomas, colorectal cancer, pancreatic cancer, B cell malignancies, non-small cell lung carcinoma (NSCLC) and ovarian cancer (Tanese K, et al., Am J Pathol. 2008;173(3): 835-43; Carmon, K. S. et al., Proc Natl Acad Sci USA., 2011; 108, 11452-7; Jiang XM, et al. Proc Natl Acad Sci USA. 2013;110(31):12649-54; Gao et al. Trans! Cancer Res., 2019; 8(1): 203-211 and McClanahan T, et al., Cancer Biol Ther. 2006;5(4): 419-26).

Primarily Lgr5 upregulation in cancer has been demonstrated to stimulate cancer stem cell proliferation and renewal as well as promoting cancer mobility and tumour formation. Lgr5 has also been shown to promote epithelial to mesenchymal transition in breast cancer (Yang L, et al., Stem Cells. 2015;33(10): 2913-24.). As such, Lgr5 has an important role in cancer metastasis and secondary tumour formation and is expressed on cancer stem cells (CSC).

The present invention is predicated, in part, on the recognition by the inventors that cells expressing chimeric antigen receptors directed against Lgr5 kill Lgr5 expressing cells and therefore can provide an alternative treatment for patients suffering from cancer.

Accordingly, the present invention provides a chimeric antigen receptor

including, or consisting of: an extracellular domain including a binding domain which recognises Lgr5; a transmembrane domain; and an intracellular signalling domain that activates a cellular function.

Throughout the specification, the CAR constructs of the present invention (collectively referred to as CNA CAR family constructs) will be referred to as CNA30xx or CNA31)(x, with the suffix “xx” being a series number (namely, 02, 03 or 04). As such the six CNAs exemplified herein are CNA3002, CNA3003, CNA3004, CNA3102, CNA3103 and CNA3104. The prefix CNA30xx refers to CARs having a first orientation of a variable light chain and a variable heavy chain in the binding region while CNA31xx have the reverse orientation. The series numbers (“)o(”) refer to varying linker lengths.

Chimeric antigen receptors (CARs) are artificially constructed proteins that upon expression on the surface of a cell can induce an antigen-specific cellular response. In their most basic form CARs include at least three domains. The first domain being an extracellular antigen-recognition domain that specifically recognises an antigen, or more specifically an epitope portion, or portions, of an antigen. The second domain being an intracellular signalling domain that is capable of inducing, or participating in the induction, of an intracellular signalling pathway. And the third domain being a transmembrane domain that traverses the plasma membrane and bridges the extracellular antigen-recognition domain and the intracellular signalling domain.

The combination of the first two domains determines the antigen specificity

of the CAR and the ability of the CAR to induce a desired cellular response, the latter of which is also dependent on the host cell of the CAR. For example, the activation of a CAR expressed in a T-helper cell and having a signalling domain comprising a CD3 activation domain may — once activated by its cognate antigen — induce the CD4+T- helper cell to secrete a range of cytokines. In a further example, the same CAR when expressed in a CD8+cytotoxic T cell — once activated by a cell expressing the cognate antigen — may induce the release of cytotoxins that ultimately lead to the induction of apoptosis of the antigen-expressing cell.

The third domain (the transmembrane domain) may comprise a portion of, or may be associated with, the signalling domain of the CAR. The transmembrane 5 domain is typically one or more hydrophobic helices, which spans the lipid bilayer of a cell and embeds the CAR within the cell membrane. The transmembrane domain of the CAR can be one determinant in the expression pattern of the CAR when associated with a cell. For example, using a transmembrane domain associated with a CD3 co- receptor can permit expression of the CAR in naïvecells, amongst others, whilst use 10 of a transmembrane domain from a CD4 co-receptor may direct expression of a CAR in T-helper cells. Use of the CD8 co receptor transmembrane domain may direct expression in cytotoxic T lymphocytes (CTLs), while the CD28 transmembrane domain may permit expression in both CTLs and T helper cells and can assist in stabilising the CAR.

15 A further component, or portion, of a chimeric antigen receptor may be a

linker domain. The linker domain spans from the extracellular side of the transmembrane domain to the antigen-recognition domain, thereby linking the antigen- recognition domain to the transmembrane domain. Typically, in the art, the linker domain is considered as an optional domain, as some CARs function without a linker domain.

Binding Domain

As used throughout the specification the term “recognises” (in relation to Lgr5) refers to the ability of the binding domain to associate with a desired epitope of Lgr5 or to any portion of the Lgr5 molecule. Preferably this recognition is selective, in 25 that the binding domain binds exclusively, or predominantly, to Lgr5. In some embodiments, the binding domain may directly bind to Lgr5, or an epitope thereof. In some embodiments, the antigen-recognition domain may bind to a processed form of Lgr5. As used in this context, the term “processed form” relates to forms of Lgr5 which have been truncated or digested, typically, as a result of intracellular processing 30 including forms and epitopes of Lgr5 which are presented on major histocompatibility complexes (e.g. human leukocyte antigens).

The CAR binding domain can be any suitable domain that can recognise Lgr5, or an epitope thereof. As used throughout the specification the term “binding domain” refers to the portion of the CAR that provides the specificity of the CAR for Lgr5. The binding domain, in the context of the present invention, only comprises a portion of the extracellular region (or ectodomain) of the CAR.

Leucine-rich repeat-containing G-protein coupled receptor 5 (SEQ ID No. 1 — Uniprot accession number 075473, NCBI Accession number NP_003658.1 and NM 003667.2 — also known by the synonyms GPR49, GPR67, FEX and GPR HG38) is a 907 amino acid long membrane bound receptor. Lgr5 consists of an extracellular domain of 561 amino acids including a 21 amino acid signalling domain and up to 17 leucine-rich repeats from amino acid 67-446. The leucine-rich repeats form an arcuate structure with a convex surface in the extracellular domain and a concave surface which interacts with RSP01 and RNF43 (Kumar K. et al., Protein Sci. 2014; 23(5): 551-65 Chen P. et al. Genes Dev., 2013; 27(12): 1345-50). Lgr5 further includes 7 transmembrane helical domains of 21 amino acids and an 84 amino acid cytoplasmic tail. The most c-terminal region of extracellular domain contains a hinge domain spanning resides 481-552.

Accordingly, in some embodiments, the binding domain recognises an epitope between residue 22-561. In some embodiments the binding domain recognises an epitope of Lgr5 on the convex surface of Lgr5. In some embodiments, the binding domain recognises an epitope between residues 67-446.

The binding domain of the CAR can comprise a range of binding molecules. These include antibodies (including non-conventional antibodies, such as heavy chain antibodies), antibody fragments, and protein binding scaffolds.

In some embodiments the antigen binding domain includes a binding portion

of an antibody which recognises Lgr5. In some embodiments, the binding domain includes the variable heavy chain of an antibody that binds to Lgr5. In some embodiments, the binding domain includes the variable light chain of an antibody that binds to Lgr5.

Antibodies which recognise Lgr5 are known in the art and include anti-Lgr5

antibodies available from: Huabio (ET1608-18), LifeSpan BioSciences (LS-A1236), mybiosource (MBS856950, MBS7113118 and MBS9604330), Novus Biologicals (NLS1236), Biorbyt (orb137136 and orb137177), ProSci (23-394), BioVision (A1007- 100), Cusabio (CSB-PA012906LAO1HU), StressMarq Biosciences Inc (SPC-764), Biolegend (373803 and 373804), R&D systems (MAB8240), Bioss (bsm-52412R), Biorad (AHP2742), GOBiosciences (ITA3755), RayBiotech (144-10545-50), Atlas

Antibodies (HPA012530), OriGene Technologies (TA890013), Elabscience (E-AB- 63432), GeneTex (GTX71143), St John's Laboratory (STJ112563), ThermoFisher (MA5-32978 and MA5-32979), NSJ Bioreagents (F48166), Acam (ab219107), Abnova (PAB30456 and PAB2591), Miltenyi Biotec (130-111-390), Sino Biological (100687- T04), Abbexa Ltd (abx019126), MBL International (MC-1236 and MC-1235), BosterBio

(M00239), Signalway Antibody (21659 and C44974), Wuhan Fine Biotech (FNab04765), Merck (HPA012530), Leading Biology (AMM03069G), Neuromics (RA25071), Antibody Research Corporation (111302), Bon Opus Biosciences (BA111499), Creative Diagnostics (L640) and Creative Biolabs (NAB-1650-VHH). Further anti-Lgr5 antibodies are disclosed in US20190248889A1 (antibodies

GEN89.LGR5.1-12 and antibodies GEN89.LGR5.26-1).

In some embodiments, the antibody is a humanised antibody. In preferred embodiments, the antibody which recognises Lgr5 is 18G7H6A3 or 18G7H6A1 (BNC101) as described in W02015153916 - entitled: Humanized antibodies that bind Igr5 and W02018232164A1 - entitled: Antibody drug conjugates that bind Igr5 (the entire disclosures of which are hereby incorporated in their entirety). Accordingly, in some embodiments the binding domain of the CAR of the present invention includes a binding portion of the antibody 18G7H6A1 or 18G7H6A3.

Epitope mapping of the interaction of antibodies 18G7H6A1 and 18G7H6A3 with Lgr5 using hydrogen-deuterium exchange mass spectrometry (see Morris, 1996

“Epitope Mapping Protocols,” in Methods in Molecular Biology vol. 66; Humana Press, Totowa, N.J.) identified the critical epitope residues as T175, E176, Q180, R183, S186, A187, Q189, D247, E248, T251, R254, S257, N258, and K260 of Lgr5 (SEQ ID No. 1). These residues are within the convex surface of leucine rich repeats 6 to 9. Therefore, in some embodiments the binding domain of the CAR recognises an epitope within leucine rich repeats 6 to 9 of Lgr5. In some embodiments, the binding domain of the CAR recognises one or more epitope residues T175, E176, Q180, R183, S186, A187, Q189, D247, E248, T251, R254, S257, N258, and K260 of Lgr5.

Antibodies 18G7H6A3 or 18G7H6A1 are humanised and affinity matured versions of the murine antibody 18G7.1. By performing alanine scanning mutagenesis individual residues in the heavy chain CDR1 and the light chain CDR1 and CDR3 were mutated to alanine, transfected into HEK 293 cells, and the resultant conditioned media was assayed for LGR5 antigen binding activity by flow cytometry. Saturation mutagenesis was performed on the heavy chain CDR3, in which every residue in CDR3 was mutated to each of the 19 naturally occurring amino acids except the original amino acid identity at that position. Each of the mutants were transfected into HEK 293 cells, and the resultant conditioned media was assayed for LGR5 antigen binding activity by flow cytometry. Comparable mutagenesis can be employed on the CD2 sequences of the heavy and light chain.

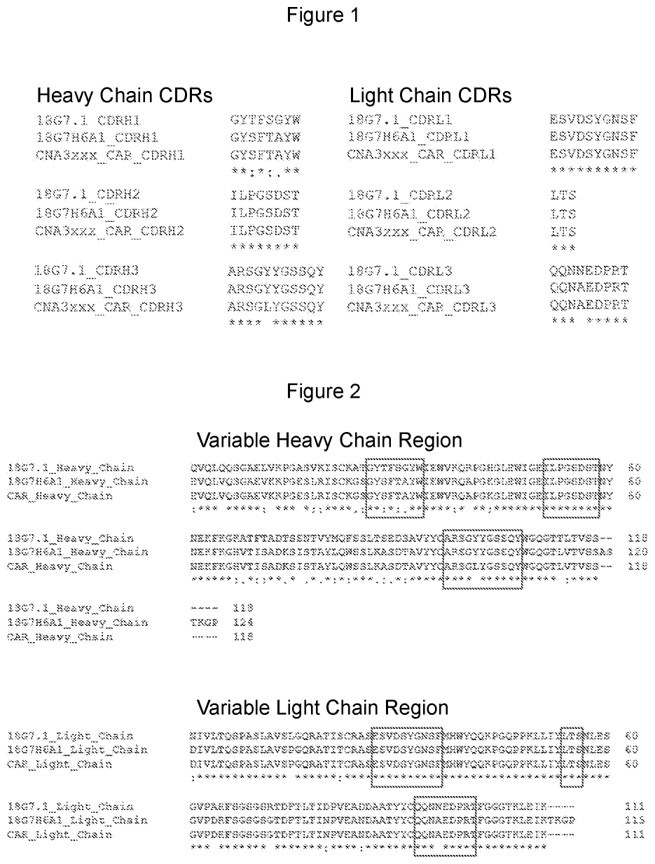

FIG. 1 illustrates the alignment of the CDR regions of 18G7.1, 18G7H6A1 and the CDRs of the present CAR construct of the present invention. As can be seen, up to three amino acids residues are different in the heavy chain CDR1 between 18G7.1 and the CAR construct of the present invention.

Further, FIG. 2 illustrates the alignment of the heavy chain variable regions of 18G7.1, 18G7H6A1 and CNA3xxx family of CARs (absent the leader sequences). As can be seen, the heavy chain variable region of the present invention differs from 18G7H6A1 with a Y to L substitution at position 101 (see FIG. 2). A comparison of the heavy chain CDR3 region of the CNA3xxx CAR (SEQ ID No. 39) and that of the antibody 18G7H6A1 (SEQ ID No. 96) illustrates that this modification is within the CDR3 region of the heavy chain (see FIG. 1)

As illustrated in FIG. 1, the light chain CDR3 regions of the 18G7.1, 18G7H6A1 and CNA3xxx CARs shows that 18G7.1 includes an N to A mutation at position 4 (c.f. SEQ ID No. 97).

In some embodiments, the binding domain includes: a heavy chain CDR1 having an amino acid sequence as set forth in SEQ ID No. 37 or having an amino acid sequence as set forth in SEQ ID No. 37 with up to 1, 2 or 3, amino acid modifications, a heavy chain CDR2 having an amino acid sequence as set forth in SEQ ID No. 38, or having an amino acid sequence as set forth in SEQ ID No. 38 with up to 1, 2 or 3 amino acid modifications, and a heavy chain CDR3 having an amino acid sequence as set forth in SEQ ID No. 39, or having an amino acid sequence as set forth in SEQ ID No. 39 with up to 1, 2 or 3 amino acid modifications.

In some embodiments, the binding domain includes: a heavy chain CDR1, CDR2 and CDR3 having an amino acid sequence as set forth in SEQ ID No. 37, 38 and 39.

In some embodiments, the binding domain includes a heavy chain CDR1 having SEQ ID No. 95, and any of the heavy chain CDR2 or CDR3 set forth in SEQ ID Nos. 38, 39 or 96.

In some embodiments, the binding domain includes a heavy chain CDR3 having SEQ ID No. 96, and any of the heavy chain CDR1 or CDR2 set forth in SEQ ID

Nos. 37, 95 or 38.

In some embodiments, the binding domain includes: a light chain CDR1 having an amino acid sequence set forth in SEQ ID No. 40 or having an amino acid sequence as set forth in SEQ ID No. 40 with up to 1, 2 or 3 amino acid modifications, a light chain CDR2 having an amino acid sequence set forth in SEQ ID No. 41 or having an amino acid sequence as set forth in SEQ ID No. 41 with up to 1, 2 or 3 amino acid modifications, and a light chain CDR3 having an amino acid sequence set forth in SEQ ID No. 42 or having an amino acid sequence as set forth in SEQ ID No. 42 with up to 1, 2 or 3 amino acid modifications.

In some embodiments, the binding domain includes: a light chain CDR1,

CDR2 and CDR3 having an amino acid sequence set forth in SEQ ID No. 40, 41 and 42.

In some embodiments, the binding domain includes a light chain CDR3 having an amino acid sequence set forth in SEQ ID No. 97, and a CDR1 and CDR2 having SEQ ID Nos. 41 and 42.

Antibodies and Antibody Fragments

As discussed above, the binding domain of the CAR may comprise an antibody or a binding portion of, or derived from, an antibody. Antibodies are protein binding molecules that have exemplary diversity with potentially as many as 10 11 to

RECTIFIED SHEET (RULE 91) ISA/AU 10 12 unique molecules in a single individual, with genetic variation between individuals allowing for further diversity. Antibody diversity in vivo is driven by random recombination of a series of genes in V(D)J joining.

The binding of an antibody is determined primarily by the three hypervariable 5 regions of the heavy and light chain, termed complementarity-determining regions

(CDR) 1, 2 and 3. As such, each mature antibody has six CDRs (variable heavy (VH) chain CDR1, CDR2, and CDR3 and variable light (VL) chain CDR1, CDR2 and CDR3). These hypervariable regions form the three-dimensional antigen-binding pocket, with the binding specificity of the antibody determined by the specific amino acid sequences 10 in the CDRs, primarily CDR3.

Antibodies to specific analytes may be obtained commercially or generated by methods known in the art. For example, antibodies to specific analytes may be prepared using methods generally disclosed by Howard and Kaser (Making and Using Antibodies: a Practical Handbook, CRC Press, 2007).

15 The specificity, avidity and affinity of antibodies generated within subjects

can be modified by way of in vitro processes such as affinity maturation (see for example; Fujino Y. et al. Biochem Biophys Res Comm., 2012; 428(3): 395-400; Li, B. et al. MAbs. 2014; 6(2): pp.437-45 and Ho M and Pastan I, “In vitro Antibody Affinity Maturation Targeting Germline Hotspots”, Method Mol Biol., 2009; 525:293-xiv). These 20 techniques include (but are not limited to) site-directed mutagenesis and PCR-driven mutagenisis, phage library development and affinity screening. For example, mutation adjacent to hotspot locations defined by A/G-G-C/T-A/T (RGYW) and AG-C/T (AGY) sequences (with reference to the coding immunoglobulin DNA) are likely to modify the affinity of produced antibodies. Alternatively, processes such as in vitro scanning 25 saturation mutagenesis (Chen, G et al. Protein Eng Des Sel., 1999; (12)4: 346-356) can be used to replace each and every modification within a CDR region with each other possible mutation. Each variant then assessed for antigen affinity and specificity. As such, in vivo derived antibodies, or binding fragments thereof, can be further modified to produce distinct, yet lineally related, antibodies. Consequently, the term 30 “antibody” (and fragments thereof) encompasses in vivo derived antibodies and in vitro derived molecules that have undergone processes of mutation to modify the CDR binding sites, such that they have unique sequences when compared to the antibodies generated in vivo. Further, binding portions of antibodies, in particular the CDRs, can be affinity matured and mutated using techniques known in the art.

The term antibody also includes non-conventional antibodies generated from species such as camelids, shark and jawfish. As such, the term antibody includes heavy-chain antibodies including camelid antibodies, IgNARs and variable lymphocyte receptors (VLRs). Further, these can be fragmented into their biding portions (such as VNARs — single binding portion of IgNARs) or integrated recombinantly into a fusion protein. Methods are known in the art for generating and adapting such non- conventional antibodies, for example see Nuttall, S., Methods Mol. Biol, 2012; 911: pp.27-36 and Vincke C. et al., Methods Mol. Biol. 2012; 907: pp.145-76.

Antibodies can be generated which bind the specifically identified epitope regions of Lgr5. Further, such antibodies can be affinity matured to optimise abiding affinity and avidity. Therefore, in some embodiments the binding domain includes a sequence identical to the binding region(s) of an antibody that binds to Lgr5, or includes a sequence corresponding to an affinity matured form of the binding region that binds to Lgr5. While affinity matured binding regions can significantly vary from the original antibody binding regions, in preferred forms the affinity mature form of the binding region has 80%, 85%, 90%, 95% 97%, 98% or 99% or greater sequence identity to an antibody that binds to Lgr5. Accordingly, the binding domain of the CAR may comprise a sequence having 80%, 85%, 90%, 95% 97%, 98% or 99% or greater sequence identity to an antibody that binds to Lgr5.

Antibody binding fragments

In some embodiments, the binding domain is an antibody binding fragment. Antibody binding fragments can be derived from an antibody or may be recombinantly generated with sequences identical to the CDRs of an antibody or antibody fragment.

Indeed, these CDRs may be from an affinity matured antibody and therefore may not be identical to an in vivo derived antibody.

Antibodies are comprised of four chains (two heavy and two light chains) and can be separated into the Fc (fraction crystallisable) and the Fab (fraction antigen binding) domains. The Fc portion of the antibody interacts with Fc receptors and the complement system. Consequently, the Fc portion is important for the immune function of the antibody. However, the Fab portion contains the binding regions of the antibody and is critical for the specificity of an antibody for the desired epitope.

Accordingly, in some embodiments, the binding domain is a Fab fragment of an antibody. Fab fragments can be individual Fab fragments (i.e. the antibody fragment is generated in the absence of linking disulphide bridges) or an F(ab')2 fragment which comprises the two Fab fragments of an antibody linked via disulphide bridges. These fragments are typically generated by fragmenting an antibody using digestion enzymes, such as pepsin. Methods are known in the art, for example see Sjogren, J. et al., Methods Mol Biol. 2017; 1535: pp.319-329.

Each Fab fragment of an antibody has six CDRs in total with the VH and VL

chains comprising three CDRs each (within a framework consisting of 4 framework regions). The constant regions of the Fab fragment can be removed to leave only the VH and VL regions of an antibody. Individual VH and VL chains (each only comprising three CDRs) have been shown to bind specifically with high affinity. Typically, individual binding regions are known as single antibody domains (sdAbs). Alternatively, the VH and VL chains can be linked via a linker to form a fusion protein known as a single- chain variable fragment (scFv — also known as a diabody). Unlike Fabs, scFvs are not fragmented from an antibody, but rather are typically recombinantly formed based on the CDR and framework regions of an antibody. Further, sdAbs can also be recombinantly produced and form the binding component of a larger fusion protein which may also include additional portions. Consequently, in some embodiments, the binding domain is, or includes, a scFv or a sdAb. The scFv may include multiple VH and VL chains linked together to form a multivalent scFv, such as a di-scFv or a tri- scFv.

Therefore, in some embodiments, the binding domain is a single-domain

antibody (sdAb) including a sequence identical to a variable heavy or variable light chain of an antibody that binds to Lgr5, or a sequence identical to a fragment antigen binding (Fab) fragment of an antibody that binds to Lgr5, or a sequence identical to a single-chain variable fragment (scFv) comprising the variable heavy and variable light regions of an antibody that binds to Lgr5.

In some embodiments, the variable heavy region has the amino acid sequence of SEQ ID No. 50, or a variant thereof having at least 80% or 90% sequence identity. In some embodiments, the variable light region has the amino acid sequence of SEQ ID No. 49, or a variant thereof having at least 80%, or 90% sequence identity.

In some embodiments, the binding domain is a single-chain variable

fragment which includes the C-terminus of a VH chain linked to the N-terminus of a VL chain, wherein the VH chain includes; a CDR1 having the sequence of SEQ ID No. 37, a CDR2 having the sequence of SEQ ID No. 38, and a CDR3 having the sequence of SEQ ID No. 39, and the VL chain includes; a CDR1 having the sequence of SEQ ID No. 40, a CDR2 having the sequence of SEQ ID No. 41, and a CDR3 having the sequence of SEQ ID No. 42, and wherein each CDR can have 1, 2 or 3 amino acid modifications.

In some embodiments, the binding domain is a single-chain variable fragment which includes the C-terminus of the VL chain linked to the N-terminus of the VH chain, wherein the VL chain includes; a CDR1 having the sequence of SEQ ID No.

a CDR2 having the sequence of SEQ ID No. 41, and a CDR3 having the sequence of SEQ ID No. 42, and VH chain includes; a CDR1 having the sequence of SEQ ID No. 37, a CDR2 having the sequence of SEQ ID No. 38, and a CDR3 having the sequence of SEQ ID No. 39, and wherein each CDR can have 1, 2 or 3 amino acid modifications.

In some embodiments, the CDR1 and CDR3 regions of the variable light

chain include up to 1, 2 or 3 mutations. In some embodiments, the CDR1 and/or CDR3 regions of the heavy chain include up to 1, 2 or 3 mutations. In some embodiments, the CDR3 of the light chain includes up to 1, 2, or 3 mutations.

The variable heavy chain and variable light chain can be fused by any suitable fusion domain. In some embodiments, the fusion domain has the sequence of

SEQ ID No. 98. Accordingly, in some embodiments, the binding domain includes a sequence set forth in SEQ ID No. 53 or SEQ ID No. 54. In some embodiments, the binding domain includes variants of SEQ ID No. 53 or SEQ ID No. 54 having at least 90% sequence identity to SEQ ID No. 53 or 54. In some embodiments, the binding domain includes variants of SEQ ID No. 53 or SEQ ID No. 54 having at least 80% sequence identity to SEQ ID No. 53 or 54.

Antibodies, fragments of antibodies, or fusion proteins containing antibody derived sequences may be obtained commercially or generated by methods known in the art, such as those discussed above.

In the above context (and as used throughout this specification), the terms “sequence identity” or “identical to”, and the like, are to be interpreted as the degree of similarity between sequences of amino acids or nucleic acids. Unless qualified by a numerical percentage or range, these terms should, by default, be interpreted as requiring functional sequence identity. That is to say that the sequences should be interpreted as being identical with the exclusion of redundant modifications and mutations which do not functionally affect the sequence. Such mutations are described herein.

Throughout the specification it is to be appreciated that the term “derived from” is not a reference to the source of a polypeptide or nucleotide per se, but rather refers to the derivation of the sequence information that constitutes a portion of the polypeptide or nucleotide such as portions of, or the entirety of, the binding domain, or intracellular signalling domain. Consequently, the term “derived from” includes synthetically, artificially or otherwise created polypeptides or nucleotides that have sequence identity to the peptide or nucleic acid from which they were derived.

Protein Binding Scaffolds

The antigen binding domain of the CAR of the present invention may be, or

may include, a protein scaffold. Protein binding scaffolds have emerged as viable molecules for binding with a diverse range of molecules and proteins. Protein binding scaffolds typically comprise a stable protein structure (scaffold) which can tolerate modification of amino acids within designated binding regions without alteration of the relative arrangement of the binding domains. These protein-binding scaffolds include (but are not limited to): Adnectins, Affilins (Nanofitins), Affibodies, Affimer molecules, Affitins, Alphabodies, Aptamers, Anticalins, Armadillo repeat protein-based scaffolds, Avimers, Designed Ankyrin Repeat Proteins (DARPins), Fynomers, Inhibitor Cystine Knot (ICK) scaffolds, Kunitz Domain peptides, Monobodies (AdNectinsTM) and Nanofitins.

Affilins are artificially created proteins of about 20kDa. They include scaffolds that are structurally related to human ubiquitin and vertebrate gamma-B crystallin, with eight surface-exposed manipulatable amino acids which and can be designed to bind specifically to target analytes. Affilin molecules can be specifically 5 adapted to biding to a large variety of molecules using techniques such as site-directed mutagenesis and phage display libraries. Methods for producing and selecting Affilin molecules are known in the art, for example, Lorey, S. et al., J Bio Chem. 2014; 289(12): pp.8493-507.

Affibodies are proteins of about 6kDa which comprise the protein scaffold of 10 the Z domain of the IgG isotype antibody with modification to one or more of 13 amino acid residues located in the binding domains of its two alpha-helices. Methods for engineering and producing affibody molecules are known in the art including Feldwisch, J. and Tolmachev, V. “Engineering of affibody molecules for therapy and diagnostics”, Methods in Molecular Biology (2012); 899: pp103-126.

15 Affimer molecules are proteins of about 12 to 14 kDa which utilise a protein

scaffold derived from the cysteine protease inhibitor family of cystatins. Affimer molecules contain two peptide loop regions in addition to an N-terminal sequence which can be adapted for target-specific binding. Affimer molecules having 1010 combinations of amino acids at the binding sites can be generated using phage display 20 libraries and techniques known in the art such as Hoffmann T. et al. Protein Eng. Des.

Sel. 2010; 23 (5): pp403-13.

Affitins are proteins of 66 residues (about 7 kDa) and use a protein scaffold derived from the DNA binding protein Sac7d found in Sulfolobus acidocaldarius. They are readily produced in vitro from prokaryotic cell cultures and contain 14 binding 25 residues which can be mutated to produce in excess of 3x10 12 structural variants.

Techniques are known in the art for producing Affitins including Mouratou et al. PNAS. 2007; Nov 13; 104(46): 17983-17988. Screening techniques such as surface plasmon resonance can be used to identify specific binding of these molecules.

Alphabodies are approximately 10kDa molecules that, unlike most 30 macromolecules, can penetrate the cellular membrane and therefore can bind to intracellular and extracellular molecules. The scaffold of Alphaboides are based on computationally designed coiled-coil structures with three alpha-helices (A, B and C) which are not analogous to natural structures. Amino acids on the A and C alpha- helices can be modified to target specific antigens. Methods for generating alphabodies and screening their binding to target molecules are described in at least US20100305304A1.

Aptamers for binding proteins include a range of nucleic acids (DNA, RNA and XNA) and peptides, which can be screened for binding to specific target molecules. Databases of nucleic acid aptamer (see for example Nucleic Acids Research, Volume 32, Issue suppl_1, 1 January 2004, pp.95-100, https://doi.org/10.1093/nar/gkh094) allow for the selection of in vitro identified DNA aptamers. Peptide aptamers consist of short amino acid sequences that generally are embedded in a looped structure within a stable protein scaffold frame (a “loop on a frame”). Typically, a 5 to 20 residue peptide loop is the source of variability for selective biding to target molecules. Combinatorial libraries and techniques such as yeast-two hybrid screening can be used to generate and screen peptide aptamers. Other techniques for generating and screening of protein aptamers are known and described in the literature including, Reverdatto S. et al., Curr Top Med Chem. 2015; 15(12): pp1082-1101.

Anticalin proteins are protein binding molecules that are derived from lipocalins. Typically, anticalins bind to smaller molecules than antibodies. Methods for screening and developing anticalins are disclosed in the art including Gebauer, M. and

Skerra, A., “Anticalins: small engineered binding proteins based on the lipocalin scaffold”, Methods Enzymology, 503 (2012), pp. 157-188 and Richter, A. et al. FEBS Letters. 2014; 588(2): pp213-218.

Armadillo repeat protein-based scaffolds are characterized by an armadillo domain, composed of tandem armadillo repeats of approximately 42 amino acids, formed into a super-helix of repeating units composed of three a-helices each. Modification of residues, within the conserved binding domain, allow for preparation of a range of combinatorial libraries which can be used for selection of target-specific binders (see for example Parmeggiani F et al. J Mol Biol. 2008; 376(5): pp1282-304.)

Avimers (also known as avidity multimers, maxibodies or low-density

lipoprotein receptor (LDLR) domain A) comprise at least two linked 30 to 35 amino acid long peptides based on the A domain of range of cysteine-rich cell surface receptor proteins. Modification of the A domain allows for directed binding to a range of epitopes on the same target or across targets, with the number of linked peptides determining the number of possible targets per avimer. A range of avimer phage display libraries are known in the art including commercial libraries such as those of Creative Biolabs.

Designed Ankyrin Repeat Proteins (DARPins) are engineered binding proteins derived from ankyrin proteins. Methods are known in the art for screening and identifying DARPins, for example, Stumpp MT, et al., Drug Discov. Today 2008; 13(15- 16): pp695-701 and Pluckthurn A., Annu. Rev. in Pharmacol. Toxicol., 2015; 55: pp489- 511.

Inhibitor Cystine Knot (ICK) scaffolds are a family of miniproteins (30 to 50 amino acid residues long) which form stable three-dimensional structures comprising three disulphide bridges connecting a series of loops having high sequence variability. Inhibitor Cystine Knots include three family members being knottins; cyclotides and growth factor cysteine-knots. Databases are known in the art, such as the KNOTTIN database (www.dsimb.inserm.fr/KNOTTIN/) which disclose specific properties of known Knottins and cyclotides, such as their sequence, structure and function. Further, methods for producing ICKs and screening for binding are known including Moore, S. et al., Protein Engineering for Therapeutics, Part B — Chapter 9, Methods in Enzymology (2012), 503; pp.223-251.

Monobodies (also known under the trade name AdNectins) utilise an FN3 (fibronectiv type III domain) scaffold with diverse and manipulatable variable groups. Adnectis share antibody variable domains and a beta-sheet loop with antibodies. The binding affinity of monobodies can be diversified and customised by in vitro evolution methods such as mRNA display, phage display and yeast display. Methods for screening and producing monobodies are disclosed in the art including Park SH et al., PLoS One , 2015; 10(7) doi: 10.1002/pro.3148 and Lipovsek, D., Protein Eng. Des. Sel., 2011; 24(1-2):3-9.

Linker Domain

The linker domain connects the transmembrane domain and antigen

recognition domain of the CAR. CAR T cells have been formed that function without the inclusion of a linker domain, and therefore, in this context, a linker domain is not considered to be generally essential to the function of all CARs.

Without wanting to be bound by theory, a linker domain may provide an appropriate molecular length to the ectodomain (extracellular domain) of the CAR to allow recognition of the epitope by the antigen recognition domain, while forming the correct immunological synaptic distance between the effector cell expressing the CAR, and the target cell. Further, the linker domain may provide the appropriate flexibility for the antigen recognition domain to be orientated in the correct manner to recognise its epitope.

Therefore, in some embodiments, the extracellular domain includes a linker

domain which links the binding domain to the transmembrane domain. In some embodiments, the linked domain is at least 12 amino acids in length. In some embodiments, the linked domain is at least about 12 amino acids in length. In some embodiments, the linked domain is greater than 12 amino acids in length. In some embodiments, the linked domain is at least 119 amino acids in length. In some embodiments, the linked domain is at least about 119 amino acids in length. In some embodiments, the linked domain is greater than 119 amino acids in length. In some embodiments, the linked domain is at least 229 amino acids in length. In some embodiments, the linked domain is at least about 229 amino acids in length. In some embodiments, the linked domain is greater than 229 amino acids in length

In some embodiments, the linked domain is up to 119 amino acids in length. In some embodiments, the linked domain is up to about 119 amino acids in length. In some embodiments, the linked domain is up to 229 amino acids in length. In some embodiments, the linked domain is up to about 229 amino acids in length.

The selection of a suitable linker domain may be based on (i) reducing

binding affinity to Fc Receptors (such as the Fcy and FcRn receptor), which minimizes ‘off-target’ activation of CAR expressing cells and (ii) optimizing the efficacy of the CAR construct by enhancing the flexibility of the antigen binding region, reducing spatial constraints for formation of an immune synapse (e.g. reducing steric hindrance and optimising synaptic distance).

In some embodiments, the linker domain includes a sequence identical to a hinge region from an immunoglobulin, or a hinge or extracellular region from a membrane bound molecule involved in the formation of a T cell synapse. For example, the linker domain may comprise a region having an amino acid sequence homologous to a hinge region from CD4, CD8, CD3, CD7 or CD28.

In some embodiments, the linker domain includes a sequence identical to a portion of an immunoglobulin. In some embodiments, the portion is one or more of a hinge region (for example the IgG4 hinge region or a modified version thereof), a constant heavy (CH)1 region, a CH2 region, a CH3 region or a CH4 region. In some embodiments, the portion is a CH2 region, a CH3 region or a hinge region of an immunoglobulin or has at least 50%, 60%, 70%, 80%, 90%, 95%, 98% or 99% sequence identity with said CH regions. In some embodiments, the portion is a CH2 region or a CH3 region and a hinge region of an immunoglobulin. In some embodiments, the immunoglobulin is selected from the IgG subtype.

In some embodiments, the linker domain includes a sequence having

similarity to a portion of one or more of IgG1, IgG2, IgG3 or IgG4 Fc regions, for example the IgG1 hinge region and the CH2 or CH3 regions of IgG4 or functional variants thereof having at least 50%, 60%, 70%, 80%, 90%, 95%, 98%, 99% or 99.5% sequence identity.

In some embodiments, the linker domain includes all, or part of, an

immunoglobulin hinge region. As would be understood in the art, the specific region that forms the hinge region of an immunoglobulin varies for different isotypes. For example, IgA, IgD and IgG isotype immunoglobulins have a hinge region between the CH1 and CH2 regions, while the function of the hinge region is provided by the CH2 region in IgE and IgM isotype immunoglobulins.

In at least some embodiments of the CAR of the present invention the linker domain includes an amino acid sequence identical to the sequence of the IgG4 hinge or includes a sequence identical to a modified version of the IgG4 hinge, preferably having 1, 2 or 3 amino acid modifications.

In some embodiments comprising a binding domain including a single-chain

variable fragment formed by the C-terminus of a variable light chain linked to the N- terminus of a variable heavy chain, wherein the variable light chain includes or consists of SEQ ID No. 49 (or a functional variant thereof) and the variable heavy chain includes or consists of SEQ ID No. 50 (or a functional variant thereof), the linker regions is at least 119 amino acids in length, or is greater than 119 amino acids in length, or at least 5 229 amino acids in length, and links the binding domain to the transmembrane domain, and wherein the linker domain is linked to the C-terminus of the variable heavy chain.

In some embodiments the binding domain comprises the C-terminus of a variable light chain linked to the N-terminus of a variable heavy chain, wherein the variable light chain includes CDRs having the amino acid sequences of SEQ ID No. 40 10 (CDR1 - ESVDSYGNSF), SEQ ID No. 41 (CDR2 - LTS) and SEQ ID No. 42 (CDR3 -

QQNAEDPRT), or functional variants thereof, and the variable heavy chain includes CDRs having the amino acid sequences of SEQ ID No. 37 (CDR1 - GYSFTAYVV), SEQ ID No. 38 (CDR2 - ILPGSDST) and SEQ ID No. 39 (CDR3 - ARSGLYGSSQY), or functional variants thereof, and the linker region is 12 amino acids, or is at least 12 15 amino acids in length, or is about 12 amino acids in length. In some forms of this embodiment the linker is up to 119 amino acids, or up to about 119 amino acids. In some alternative forms of this embodiment the linker is up to 229 amino acids, or up to about 229 amino acids. For the avoidance of doubt, the linker length can be 12 or greater, 12 to 119, or 12 to 229 amino acids in length.

20 In some embodiments the binding domain comprises the C-terminus of a

variable light chain linked to the N-terminus of a variable heavy chain, wherein the variable light chain includes CDRs having the amino acid sequences of SEQ ID No. 40 (CDR1), SEQ ID No. 41 (CDR2) and SEQ ID No. 42 (CDR3), or functional variants thereof, and the variable heavy chain includes CDRs having the amino acid sequences of SEQ ID No. 37 (CDR1), SEQ ID No. 38 (CDR2) and SEQ ID No. 39 (CDR3), or functional variants thereof, and the linker region is 119 amino acids, or is at least 119 amino acids in length, or is about 119 amino acids in length. In some forms of this embodiment the linker is up to 229 amino acids, or up to about 229 amino acids.

In some embodiments the binding domain comprises the C-terminus of a 30 variable light chain linked to the N-terminus of a variable heavy chain, wherein the variable light chain includes CDRs having the amino acid sequences of SEQ ID No. 40 (CDR1), SEQ ID No. 41 (CDR2) and SEQ ID No. 42 (CDR3), or functional variants thereof, and the variable heavy chain includes CDRs having the amino acid sequences of SEQ ID No. 37 (CDR1), SEQ ID No. 38 (CDR2) and SEQ ID No. 39 (CDR3), or functional variants thereof, and the linker region is 229 amino acids, or is at least 229 amino acids in length, or is about 229 amino acids in length.

In some embodiments the binding domain comprises the C-terminus of a

variable heavy chain linked to the N-terminus of a variable light chain, wherein the variable heavy chain includes CDRs having the amino acid sequences of SEQ ID No. 37 (CDR1), SEQ ID No. 38 (CDR2) and SEQ ID No. 39 (CDR3), or functional variants thereof and the variable light chain includes CDRs having the amino acid sequences of SEQ ID No. 40 (CDR1), SEQ ID No. 41 (CDR2) and SEQ ID No. 42 (CDR3), or functional variants thereof, and the linker region is 12 amino acids, or is at least 12 amino acids in length, or is about 12 amino acids in length. In some forms of this embodiment the linker is up to 119 amino acids, or up to about 119 amino acids. In some alternative forms of this embodiment the linker is up to 229 amino acids, or up to about 229 amino acids. For the avoidance of doubt the linker length can be 12 or greater, 12 to 119, or 12 to 229 amino acids in length.

In some embodiments the binding domain comprises the C-terminus of a variable heavy chain linked to the N-terminus of a variable light chain, wherein the variable heavy chain includes CDRs having the amino acid sequences of SEQ ID No. 37 (CDR1), SEQ ID No. 38 (CDR2) and SEQ ID No. 39 (CDR3), or functional variants thereof and the variable light chain includes CDRs having the amino acid sequences of SEQ ID No. 40 (CDR1), SEQ ID No. 41 (CDR2) and SEQ ID No. 42 (CDR3), or functional variants thereof, and the linker region is 119 amino acids, or is at least 119 amino acids in length, or is about 119 amino acids in length. In some forms of this embodiment the linker is up to 229 amino acids, or up to about 229 amino acids.

In some embodiments the binding domain comprises the C-terminus of a variable heavy chain linked to the N-terminus of a variable light chain, wherein the variable heavy chain includes CDRs having the amino acid sequences of SEQ ID No. 37 (CDR1), SEQ ID No. 38 (CDR2) and SEQ ID No. 39 (CDR3), or functional variants thereof and the variable light chain includes CDRs having the amino acid sequences of

SEQ ID No. 40 (CDR1), SEQ ID No. 41 (CDR2) and SEQ ID No. 42 (CDR3), or functional variants thereof, and the linker region is 229 amino acids, or is at least 229 amino acids in length, or is about 229 amino acids in length.

A non-exhaustive list of sequences which may be incorporated into the linker domain is provided in Table 1, below. In some embodiments, the linker domain of the present invention may include any one or more of the components provided in Table 1.

In some embodiments, the linker domain may consist of any one or more of the linkers provided in Table 1. Further, the linker domain may be an artificially synthesized sequence such poly-Glycine sequences or repeats of GGGGS (Gly4Ser) sequences (for example a (Gly4Ser)3).

TABLE 1
Possible linker domain components

| | | | SEQ |
|---|---|---|---|
| Name | length | Sequence | ID |
| IgG1 | 15 | EPKSCDKTHTCPPCP | 2 |
| hinge | | | |
| IgG2 | 12 | ERKCCVECPPCP | 3 |
| hinge | | | |
| IgG3 | 62 | LKTPLGDTTHTCPRCPEPKSCDTPPPCPRC | 4 |
| hinge | | PEPKSCDTPPPCPRCPEPKSCDTPPPCPRC | |
| | | P | |
| IgG4 | 12 | ESKYGPPCPSCP | 5 |
| hinge | | | |
| IgG4 | 12 | ESKYGPPCPPCP | 6 |
| hinge | | | |
| (mu- | | | |
| tated) | | | |
| IgA1 | 21 | PVPSTPPTPSPSTPPTPSPSC | 7 |
| hinge | | | |
| IgD | 58 | ESPKAQASSVPTAQPQAEGSLAKATTAPAT | 8 |
| hinge | | TRNTGRGGEEKKKEKEKEEQEERETKTP | |
| IgM | 112 | IAELPPKVSVFVPPRDGFFGNPRKSKLICQ | 9 |
| common | | ATGFSPRQIQVSWLREGKQVGSGVTTDQVQ | |
| heavy | | AEAKESGPTTYKVTSTLTIKESDWLGQSMF | |
| (CH) 2 | | TCRVDHRGLTFQQNASSMCVPD | |
| IgE | 99 | PTVKILQSSCDGGGHFPPTIQLLCLVSGYT | 10 |
| CH2 | | PGTINITWLEDGQVMDVDLSTASTTQEGEL | |
| | | ASTQSELTLSQKHWLSDRTYTCQVTYQGHT | |
| | | FEDSTKKCA | |
| CD28 | 15 | KHLCPSPLFPGPSKP | 11 |
| Hinge | | | |
| CD8a | 48 | AKPTTTPAPRPPTPAPTIASQPLSLRPEAC | 12 |
| Hinge | | RPAAGGAVHTRGLDFACD | |
| (1) | | | |
| CD8a | 45 | TTTPAPRPPTPAPTIASQPLSLRPEACRPA | 13 |
| Hinge | | AGGAVHTRGLDFACD | |
| (2) | | | |
| CD8b | 38 | SVVDFLPTTAQPTKKSTLKKRVCRLPRP | 14 |
| Hinge | | ETQKGPLCSP | |
| (1) | | | |
| CD8b | 36 | VDFLPTTAQPTKKSTLKKRVCRLPRP | 15 |
| Hinge | | ETQKGPLCSP | |
| (2) | | | |
| CD7 | 36 | APPRASALPAPPTGSALPDPQTASALPDPP | 16 |
| | | AASALP | |
| CD4 | 23 | DSGQVLLESNIKVLPTWSTPVQP | 17 |
| Hinge | | | |
| PolyG/S | 5+ | (GGGGS)ₙ | 18 |

In some embodiments, the linker domain includes a sequence set forth in any one or more of the sequences selected from SEQ ID Nos: 2 to 30, or a functional variant, or portion thereof, having at least 50%, 60%, 70%, 80%, 90%, 95%, 98%, 99% or 99.5% sequence identity.

In some embodiments, the linker domain includes a sequence identical to an immunoglobulin CH3 domain, an immunoglobulin CH2 domain or both a CH2 and CH3 domain. In some embodiments, the linker domain includes a sequence identical to an immunoglobulin hinge region and one or more of a CH3 domain or a CH2 domain. In some embodiments the CH2 and/or CH3 regions are from the IgG4 subclass of IgG antibodies.

In some embodiments, the linker domain includes, or consists of, a sequence selected from the group consisting of: SEQ ID No. 55, SEQ ID No. 56 or SEQ ID No. 57, or a functional variant, or portion, thereof having at least 50%, 60%, 70%, 80%, 90%, 95%, 98%, 99% or 99.5% sequence identity.

The hinge region, CH2 and CH3 region of immunoglobulins, in particular IgG isotype antibodies, may be bound by Fc receptors such as Fc gamma receptors and Fc neonatal receptors. Binding of the linker domain of a chimeric antigen receptor can reduce the efficacy of the receptor and can lead to off-target killing. Therefore, in some embodiments, the linker domain is designed such that it has a reduced, or no, capacity to bind with an Fc receptor. In some embodiments, the linker domain is identical to an immunoglobulin with a reduced capacity to bind with an Fc receptor compared to other immunoglobulin isotypes. In some embodiments, the linker domain of the chimeric antigen receptor does not comprise an amino acid sequence that substantially binds with an Fc receptor.

The ability for Fc receptors to bind with different IgG isotypes is presented in Table 2 below.

TABLE 2

| Fc Receptor binding to IgG subtypes | | | | |
|---|---|---|---|---|
| | IgG1 | IgG2 | IgG3 | IgG4 |
| FcyRIa (CD64) | +++ | − | ++++ | ++ |
| FcyRIIaa (CD32) | +++ | ++ | ++++ | ++ |
| FcyRIIba (CD32) | + | − | ++ | + |
| FcyRIIIba (CD16a) | +++ | − | ++++ | − |
| FcRn | +++ | +++ | +++ | +++ |

In some embodiments, where the linker domain includes a portion identical to the Fc region of an immunoglobulin, the portion maybe modified to reduce binding to the Fc receptor. Methods are known in the art to modify a protein to reduce binding by Fc Receptors. Fc gamma receptor primarily binds to the lower hinge region and the n- 10 terminal of the CH2 region of immunoglobulin regions, while the neonatal Fc receptor primarily binds to amino acids at the C-terminus of the CH2 region and the N-terminus of the CH3 region. A guide to the binding of Fc receptors to IgG antibodies can be found in Chapter 7 of “Antibody Fc : Linking Adaptive and Innate Immunity” Ackerman and Nimmerjahn, Elsevier Science & Technology 2014. Therefore, modifications in these 15 areas may alter the binding of Fc receptors to linker domains having homology with the

Fc portion of immunoglobulins. A non-exhaustive exemplary list of mutations to Human IgG1, which have been shown to reduce Fc-gamma receptor and FcRn binding include: E116P, L117V, L118A, G119 deleted, P121A, S122A, 1136A, S137A, R138A, T139A, E141A, D148A, 5150A, 5150A, E152A, D153A, E155A, N159A, D163A, H168A, 20 N169A, K171A, K173A, R175A, E176A, Q178A, Y179F, N180A, S181A, R184A,

V188A, T190A, L192A, Q194A, D195A, N198A, K200A, K205A, K209A, A210Q, A2105, A210G, P212A, P214A, E216A, K217A, 5220A, K221A, A222T, K243A, Q245A, H251A, D259A, A261Q, E263A, E265A, V286A, 5288A, K297A, 5307A, E313A, H316A, N317A, H318A, Y319A (numbering corresponds to the sequence set 25 forth in Uniprot reference number P01857-1).

Transmembrane and Intracellular Domains

The transmembrane domain of a CAR bridges the extracellular portion (ectodomain) to the intracellular portion (endodomain) with its role being primarily structural. As such, the transmembrane domain can consist of any sequence that can 5 anchor and span the lipid bilayer of a cell. However, the nature of the transmembrane domain can influence its localisation and expression.

In a preferred embodiment, the transmembrane domain has homology to a sequence of a molecule involved in T cell synapse formation, or T cell signal induction. In some embodiments, the chimeric antigen receptor of the present invention includes a transmembrane domain which includes a sequence identical to all, or part of, the transmembrane domain of CD3, CD4, CD8 or CD28. In some embodiments, the transmembrane domain includes a sequence having identity to all, or part of, the transmembrane domain of CD8 or CD28. In some embodiments, the transmembrane domain has sequence identity to all, or part of, the transmembrane domain of CD28. In 15 some embodiments, the transmembrane domain has sequence identity to SEQ ID No.

72, or a functional variant of the DNA or encoded amino acid sequence.

In addition to the antigen recognition domain, the linker domain and the transmembrane domain, the chimeric antigen receptor of the present invention includes an intracellular (endo) domain which includes a signalling portion (a signalling domain).

20 The intracellular signalling domain of the chimeric antigen receptor can be

any suitable domain that is capable of inducing, or participating in the induction of, an intracellular signalling cascade upon activation of the CAR as a result of recognition of an antigen by the antigen-recognition domain. The signalling domain of a CAR will be specifically chosen depending on the intended cellular outcome following activation of 25 the CAR. Whilst there are many possible signalling domains, when used in immunotherapy and cancer therapy the signalling domains can be grouped into two general categories based on the receptor from which they are derived, namely activation receptors and co-stimulatory receptors (see further details below). Therefore, in some embodiments, the signalling domain includes a portion having an amino acid 30 sequence identical to a signalling portion of an activation receptor, or a functional variant thereof. In some embodiments, the signalling domain includes a portion having an amino acid sequence identical to a signalling portion of a co-stimulatory receptor, or a functional variant.

As used throughout the specification the term “portion”, when used with respect to an activation receptor or co-stimulatory receptor, relates to any segment of the receptor that includes a sequence responsible for, or involved in, the initiation/induction of an intracellular signalling cascade following interaction of the receptor with its cognate antigen or ligand. An example of the initiation/induction of an intracellular signalling cascade for the T cell receptor (TCR) via CD3 is outlined below.

Whilst not wishing to be bound by theory, the extracellular portion of the TCR largely comprises heterodimers of either the clonotypic TCRa and TCR8 chains (the

TCRa/8 receptor) or the TCRy and TORO chains (the TCRyO receptor). These TCR heterodimers generally lack inherent signalling transduction capabilities and therefore they are non-covalently associated with multiple signal transducing subunits of CD3 (primarily CD3-zeta, -gamma, -delta, and -epsilon). Each of the gamma, delta, and epsilon chains of CD3 has an intracellular (cytoplasmic) portion that includes a single

Immune-receptor-Tyrosine-based-Activation-Motif (ITAM), whilst the CD3-zeta chain includes three tandem ITAMs. Upon engagement of the TCR by its cognate antigen in the presence of MHC, and the association of a requisite co-receptor such as CD4 or CD8, signalling is initiated which results in a tyrosine kinase (namely Lck) phosphorylating the two tyrosine residues within the intracellular ITAM(s) of the CD3 chains. Subsequently, a second tyrosine kinase (ZAP-70 — itself activated by Lck phosphorylation) is recruited to biphosphorylate the ITAMs. As a result, several downstream target proteins are activated which eventually leads to intracellular conformational changes, calcium mobilisation, and actin cytoskeleton re-arrangement that when combined ultimately lead to activation of transcription factors and induction of a T cell immune response.

As used throughout the specification the term “activation receptor” relates to receptors, or co-receptors that form a component of, or are involved in the formation of, the T cell receptor (TCR) complex, or receptors involved in the specific activation of immune cells as a result of recognition of an antigenic or other immunogenic stimulus.

Non-limiting examples of such activation receptors include components of the T cell receptor-CD3 complex (CD3-zeta, -gamma, -delta, and -epsilon), the CD4 co-receptor, the CD8 co-receptor, Fc receptors or Natural Killer (NK) cell associated activation receptors such a LY-49 (KLRA1), natural cytotoxicity receptors (NCR, preferably NKp46, NKp44, NKp30 or NKG2 or the CD94/NKG2 heterodimer).

Consequently, in some embodiments of the CAR of the present invention the signalling domain includes a portion derived from any one or more of a member of the CD3 co- receptor complex (preferably at least a signalling portion of the CD3-Zeta g) chain), the CD4 co-receptor, the CD8 co-receptor, a signalling portion of the Fc Receptor (FcR) (preferably a signalling portion of FccRI or FcyRl) or NK associated receptors such a

LY-49.

The specific intracellular signal transduction portion of each of the CD3 chains are known in the art. By way of example, the intracellular cytoplasmic region of the CD3 chain spans from amino acid 52 to amino acid 164 of the sequence set forth in SEQ ID No. 31, with the three ITAM regions spanning amino acids 61 to 89, 100 to 128 and 131 to 159 of SEQ ID No. 31. Furthermore, the intracellular portion of the CD3c chain spans amino acids 153 to 207 of the sequence set forth in SEQ ID No. 32, with the single ITAM region spanning amino acids 178 to 205 of SEQ ID No. 32. The intracellular portion of CD3y chain spans amino acids 138 to 182 of the sequence set forth in SEQ ID No. 33 with the single ITAM region spanning amino acids amino acids 149 to 177 of SEQ ID No. 33. The intracellular portion of CD3O spans amino acids 127 to 171 of the sequence set forth in SEQ ID No. 34 with the single ITAM region spanning amino acids 138 to 166 of SEQ ID No. 34.

In some embodiments of the present invention, the signalling domain includes a portion derived from, or having sequence homology to, CD3 (preferably the

CD3- chain or a portion thereof). In some embodiments, the signalling domain includes a sequence identical to all, or part of, the intracellular domain of CD3 zeta (CD3-). In some embodiments, the portion of the CD3- co-receptor complex includes the amino acid sequence set forth in SEQ ID No. 58, or a functional variant thereof having at least 50%, 60%, 70%, 80%, 90%, 95%, 98%, 99% or 99.5% sequence identity.

Alternative signalling domains include intracellular portions of the Fc receptors, which are known in the art. For example, the intracellular portions of the

FccR1 span amino acids 1 to 59, 118 to 130 and 201 to 244 of the sequence set forth in SEQ ID No. 35, or a functional variant thereof. Furthermore, the intracellular portion of FcyRl spans the amino acids 314 to 374 of the sequence set forth in SEQ ID No. 36, or a functional variant thereof.

Various combinations of portions of activation receptors can be utilized to

form the transmembrane (TM) and intracellular (IC) portions of the CAR for example the CD3 TM and CD3 IC (Landmeier S. et al. Cancer Res., 2007; 67:8335-43; Guest RD. et al., J Immunother., 2005, 28:203-11; Hombach AA. et al. J Immunol., 2007; 178: 4650-7), the CD4 TM and CD3 IC (James SE. et al. J Immunol., 2008; 180:7028-38), the CD8 TM and CD3 IC (Patel SD. et al. Gene Ther., 1999; 6: 412-9), and the FccRly

TM and the FccRly IC (Haynes NM. et al. J Immunol., 2001; 166: 182-7; Annenkov A E. et al. J Immunol., 1998; 161: 6604-13).

As discussed above, in some embodiments of the chimeric antigen receptor of the present invention the signalling domain includes a portion having an amino acid sequence identical to a signalling portion of a co-stimulatory receptor.

As used throughout the specification the term “co-stimulatory receptor” relates to receptors or co-receptors that assist in the activation of an immune cell upon antigen specific inducement of an activation receptor. As will be understood, co- stimulatory receptors do not require the presence of antigen and are not antigen specific, but are typically one of two signals, the other being an activation signal, which is required for the induction of an immune cellular response. In the context of an immune response a co-stimulation receptor is typically activated by the presence of its expressed ligand on the surface of an antigen-presenting cell (APC) such as a dendritic cell or macrophage. With specific regard to T cells, co-stimulation is necessary to lead to cellular activation, proliferation, differentiation and survival (all of which are generally referred to under the umbrella of T cell activation), whilst presentation of an antigen to a T cell in the absence of co-stimulation can lead to anergy, clonal deletion and/or the development of antigen specific tolerance. Importantly, co-stimulatory molecules can inform the T cell response to a simultaneously encountered antigen. Generally, an antigen encountered in the context of a ‘positive’ co-stimulatory molecule will lead to activation of the T cell and a cellular immune response aimed at eliminating cells expressing that antigen. Whilst an antigen encountered in the context of a ‘negative’ co-receptor will lead to an induced state of tolerance to the co-encountered antigen.

Non-limiting examples of T cell co-stimulatory receptors include CD27, CD28, CD30, CD40, DAP10, OX40, 4-1BB (CD137), ICOS. Specifically, CD27, CD28, CD30, CD40, DAP10, OX40, 4-1BB (CD137), and ICOS all represent ‘positive’ co- stimulatory molecules that enhance activation of a T cell response. Accordingly, in some embodiments of the first aspect of the present invention, the signalling domain includes a portion derived from any one or more of CD27, CD28, CD30, CD40, DAP10, OX40, 4-1BB (CD137) and ICOS.

In some embodiments of the present invention, the signalling domain

includes a portion derived from the CD28, OX40 or 4-1BB co-stimulatory receptors. In some embodiments, the signalling domain includes a portion of 4-1BB. In some embodiments, the portion of the 4-1BB co-stimulatory receptor includes the amino acid sequence set forth in SEQ ID No. 59, or a functional variant thereof.

Various portions of co-stimulatory receptors can be utilized to form the

transmembrane (TM) and intracellular (IC) portions of the CAR, alone or in combination. Examples of combinations include the CD8 TM and DAP10 IC or CD8 TM and 4-1BB IC (Marin V. et al. Exp Hematol., 2007; 35: 1388-97), the CD28 TM and the CD28 IC (Wilkie S. et al. J Immunol., 2008;180: 4901-9; Maher J. et al. Nat Biotechnol., 2002; 20: 70-5), and the CD8 TM and the CD28 IC (Marin V. et al. Exp Hematol., 2007; 35:

1388-97).

Sequence information for the above-referenced activation and co- stimulatory receptors is readily accessible in a variety of databases. For example, embodiments of human amino acid, gene and mRNA sequences for these receptors are provided in Table 3.

TABLE 3
Summary of Activation and Co-stimulation Receptor Sequence Information

| | | | GeneBank mRNA |
|---|---|---|---|
| Receptor Name | Uniprot Ref No. | NCBI Gene ID No. | Ref No. |
| CD3-zeta | P20963 | 919 | GI:166362721 |
| CD3-gamma | P09693 | 917 | GI:166362738 |
| CD3-delta | P04234 | 915 | GI:98985799 |
| CD3-epsilon | P07766 | 916 | GI:166362733 |
| CD4 | P0173 | 920 | GI:303522473 |
| CD8 alpha | P01732 | 925 | GI:225007534 |
| CD8 beta | P01966 | 926 | GI:296010927 |
| FcyRI | P12314 | 2209 | GI:31331 |
| FcER1 | Q01362 | 2206 | GI:219881 |
| Ly-49 (KLRAI) | Q7Z556 | 10748 | GI:33114184 |
| NKp46 | O76036 | 9437 | GI:3647268 |
| NKp44 | O95944 | 9436 | GI:4493701 |
| NKp30 | O14931 | 259197 | GI:5823969 |
| CD94 | Q13241 | 3824 | GI:1098616 |
| CD27 | P26842 | 939 | GI:180084 |
| CD28 | P10747 | 940 | GI:338444 |
| CD30 | P28908 | 943 | GI:180095 |
| CD40 | P25942 | 958 | GI:29850 |
| DAP10 | Q9UBK5 | 10870 | GI:5738198 |
| OX40 | P43489 | 7293 | GI:472957 |
| 4-1BB (TNR9) | Q07011 | 3604 | GI:571320 |
| ICOS | Q9Y6W8 | 29851 | GI:9968295 |
| CTLA-4 | P16410 | 1493 | GI:291928 |
| PD-1 | Q15116 | 5133 | GI:2149002 |

Whilst Table 3 is provided with reference to human activation and co- stimulatory receptors, it would be understood by a person skilled in the art that homologous and orthologous versions of each receptor are present in the majority of 5 mammalian and vertebrate species. Therefore, the above-referenced sequences are only provided as non-limiting examples of receptor sequences that may be included in a CAR of the first aspect of the present invention and homologous and orthologous sequences from any desired species may be used to generate a CAR that is suitable for the given species.

10 In some embodiments of the invention, the transmembrane domain and a

portion of the signalling domain share homology with the same molecule. For example, a portion of CD3 including the transmembrane domain and a signalling domain may be utilised. In some embodiments, the transmembrane domain includes, or consists of, a sequence identical to all or a portion of the transmembrane domain of CD28 and the signalling domain includes, or consists of, a sequence identical to all or a portion of the intracellular domain of CD28.

In some embodiments of the present invention, the signalling domain includes a portion derived from an activation receptor and a portion derived from a co- stimulatory receptor. Whilst not wishing to be bound by theory, in this context the recognition of an antigen by the antigen-recognition domain of the CAR will simultaneously induce both an intracellular activation signal and an intracellular co- stimulatory signal. Consequently, this will simulate the presentation of an antigen by an APC expressing co-stimulatory ligand. Alternatively, the CAR could have a signalling domain that includes a portion derived from either an activation receptor or a co- stimulatory receptor. In this alternative form, the CAR will only induce either an activating intracellular signalling cascade or a co-stimulatory intracellular signalling cascade.

In some embodiments of the invention the signalling domain includes, or

consists of, a sequence identical to all or a portion of the intracellular domain of 4-1BB and CD3- chain. In some embodiment, the CAR of the present invention includes an intracellular domain including, or consisting of, SEQ ID No. 59 and SEQ ID No. 58, or functional variants thereof.

In some embodiments, the CAR will have a signalling domain that includes

a portion of a single activation receptor and portions of multiple co-stimulatory receptors. In some embodiments, the CAR will have a signalling domain including a sequence identical to portions of multiple activation receptors and a portion derived from a single co-stimulatory receptor. In some embodiments, the CAR will have a signalling domain that includes a sequence identical to portions of multiple activation receptors and portions of multiple co-stimulatory receptors. In some embodiments, the CAR will have a signalling domain that includes a sequence identical to a portion of a single activation receptor and portions of two co-stimulatory receptors. In some embodiments, the CAR will have a signalling domain that includes a sequence identical to a portion of a single activation receptor and portions derived from three co- stimulatory receptors. In some embodiments, the CAR will have a signalling domain that includes a sequence identical to portions of two activation receptors, and a portion of one co-stimulatory receptor. In some embodiments, the CAR will have a signalling domain that includes a sequence identical to portions of two activation receptors and portions of two co-stimulatory receptors. As will be understood there are further variations of the number of activation receptors and co-stimulatory receptors and the above examples are not considered to be limiting on the possible combinations included herein.

In some embodiments of the invention, the sequence of the transmembrane domain and at least a portion of the signalling domain have sequence similarity to portions of distinct molecules. In some embodiments, the transmembrane domain includes, or consists of, a sequence identical to all or a portion of the transmembrane domain of CD28 and the signalling domain includes, or consists of, a sequence identical to all or a portion of the intracellular domain of 4-1BB and CD3- chain.

Chimeric Antigen Receptor

In some embodiments, the CAR will include an antigen recognition domain specific for Lgr5, a linker domain having sequence identity to the IgG4 hinge region, a transmembrane region having sequence identity to the CD28 transmembrane sequence, an intracellular portion having sequence identity to a signalling region of 4- 1BB and/or an intracellular portion having sequence identity to a signalling portion of CD3zeta, or functional variant of the described portions, domains or regions.

In some embodiments, the CAR will include an antigen recognition domain

specific for Lgr5, a linker domain having sequence identity to the IgG4 hinge region combined with the IgG4 CH3 region, a transmembrane region having sequence identity to the CD28 transmembrane sequence, an intracellular portion having sequence identity to a signalling region of 4-1BB and/or an intracellular portion having sequence identity to a signalling portion of CD3zeta, or functional variants of the described portions, domains or regions.

In some embodiments, the CAR will include an antigen recognition domain specific for Lgr5, a linker domain having sequence identity to the IgG4 hinge region combined with the IgG4 CH2 region and the IgG4 CH3 region, a transmembrane region having sequence identity to the CD28 transmembrane sequence, an intracellular portion having sequence identity to a signalling region of 4-1BB and/or an intracellular portion having sequence identity to a signalling portion of CD3zeta, or functional variants of the described portions, domains or regions.

In some embodiments of invention, the chimeric antigen receptor includes, or consists of, an amino acid sequence selected from the group consisting of: SEQ ID No. 60, 61, 62, 63, 64 or 65, or functional variants thereof. In some embodiments, the chimeric antigen receptor includes or consists of an amino acid sequence selected from the group consisting of: SEQ ID No. 62, 63, 64 or 65, or functional variants thereof.

As would be understood by a person skilled in the art modification of the CAR receptors described herein can be made without deviating from the scope of the present invention. For example, with respect to SEQ ID Nos. 60, 61, 62, 63, 64, and 65 the preferred function of the CAR is to recognise Lgr5 and induce an intracellular signal which results in the activation of a T cell expressing the CAR. As would be understood by a person skilled in the art, variation to portions of the amino acid sequence of the chimeric antigen receptor may be made without significantly altering the specificity of the CAR and/or activation of a cell (such as a T cell) expressing the CAR. Such variations may include, but are not limited to, variations in the hinge region of the chimeric antigen receptor, variations in the transmembrane domain, and variations in the portions of the activation receptors and/or co-stimulatory receptors that comprise the intracellular domain of the chimeric antigen receptor. When undertaking such variations, a person skilled in the art will utilise the knowledge and skills with the intention to arrive at a workable CAR. As such, the scope of the variations excludes those which are immediately recognisable to a person skilled in the art as resulting in the abrogation of the function of the CAR.

Nucleic Acid Constructs and Genetic Modification of Cells

The CAR described herein can be produced by any means known in the art,

though preferably it is produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

As such, the present invention further provides a nucleic acid molecule, or a nucleic acid construct including a nucleic acid molecule, including a nucleic acid sequence encoding the chimeric antigen receptor described above.

Further, the nucleic acid construct includes an expression vector including a nucleic acid sequence encoding the chimeric antigen receptor described above.

In some embodiments, the nucleic acid molecule includes a nucleotide sequence which encodes the amino acid sequence set forth in SEQ ID No. 60, 61, 62, 63, 64 or 65, or functional variants thereof.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double- stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus the term “nucleic acid molecule” embraces chemically, enzymatically, or metabolically modified forms.

In some embodiments of the invention, the nucleic acid molecule includes the nucleotide sequence set forth in SEQ ID Nos. 66, 67, 68, 69, 70 or 71, or a functional variant thereof.

To absolve doubt, it is to be understood that functional variants of SEQ ID Nos. 66, 67, 68, 69, 70 or 71 includes sequence variants having one or more different nucleic acids, but which still encode identical amino acid sequences. Because of the degeneracy of the genetic code, a large number of nucleic acids can encode any given protein. For instance, the codons GCA, GCC, GCG and GCU all encode the amino acid alanine. One of skill in the art will recognise that each codon in a nucleic acid sequence (except AUG, which is ordinarily the only codon for methionine, and TGG, which is ordinarily the only codon for tryptophan) can be modified to yield a functionally identical molecule. Accordingly, each silent variation of a nucleotide sequence that encodes a 5 polypeptide is implicit in each described sequence.

It is to be understood that a nucleic acid construct, in accordance with the invention, may further comprise one or more of: an origin of replication for one or more hosts; a selectable marker gene which is active in one or more hosts; and/or one or more transcriptional control sequences, wherein expression of the nucleic acid molecule is under the control of a transcriptional control sequence.

As used herein, the term “selectable marker gene” includes any gene that confers a phenotype on a cell in which it is expressed, to facilitate the identification and/or selection of cells, which are transfected or transduced with the construct.

“Selectable marker genes” include any nucleotide sequences which, when 15 expressed by a cell transduced with the construct, confer a phenotype on the cell that facilitates the identification and/or selection of these transduced cells. A range of nucleotide sequences encoding suitable selectable markers are known in the art (for example Mortesen, RM. and Kingston RE. Curr Protoc Mol Biol, 2009; Unit 9.5). Exemplary nucleotide sequences that encode selectable markers include: Adenosine 20 deaminase (ADA) gene; Cytosine deaminase (CDA) gene; Dihydrofolate reductase

(DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance 25 genes, hygromycin-resistance genes, kanamycin-resistance genes and ampicillin- resistance gene; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS). Further, cell selection 30 markers for T cells are specifically discussed in Barese, C.N. and Dunubar C.E., Hum.

Gene Ther., 2011; 22(6): pp.659-68. These markers include neomycin (NEO) resistance genes, NGFR (non-signalling NGFR), truncated CD34 and truncated non- signalling CD19 (ACD19). Embodiments of the present invention (as described further herein) utilise a truncated form of the epithelial growth factor receptor (EGFRt). Further techniques have been developed for tracking CAR T cells in vivo including modified eDHFD (see Sellmyer, M.A. et al. Mol. Ther., 2020; 28(1): pp.42-51).

Furthermore, it should be noted that the selectable marker gene may be a

distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence which effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid which confers, activates or enhances expression of a nucleic acid in a cell.

In some embodiments, at least one transcriptional control sequence is operably connected to the nucleic acid molecule of the second aspect of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

A promoter may regulate the expression of an operably connected nucleic

acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter which is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (E3A), phosphoglycerate kinase (PGK) and CMV early enhancer/chicken β actin (CAGG).

Inducible promoters may include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: tetracycline regulated promoters (e.g. see US Patent and US Patent 5,464,758); steroid responsive promoters such as glucocorticoid receptor promoters (e.g. see US Patent 5,512,483), ecdysone receptor promoters (e.g. see US Patent 6,379,945) and the like; and metal-responsive promoters such as metallothionein promoters (e.g. see US Patent 4,940,661, US

Patent 4,579,821 and US 4,601,978) amongst others.

As mentioned above, the control sequences may also include a terminator. The term “terminator” refers to a DNA sequence at the end of a transcriptional unit which signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues or organs in which it is intended to be used. Suitable terminators would be known to a person skilled in the art.

As will be understood, the nucleic acid construct in accordance with the

invention can further include additional sequences, for example sequences that permit enhanced expression, cytoplasmic or membrane transportation, and location signals. Specific non-limiting examples include an Internal Ribosome Entry Site (IRES), an N- terminal interleukin-2 signal peptide (Moot R. et al., Mol Ther Oncolytics, 2016; 3: 16026), CSF2RA, IgE leader sequence (W02017147458), influenza hemagglutinin signal sequence (Quitterer, U. et al., Biochem. Biophys. Res., 2011: 409(3): pp.544- 579) amongst others. A review of signal peptides is provided in Owki, H. et al. Eur. J. Cell Biol., 2018; 97(6):pp.422-441, which is herein incorporated by reference.

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

The nucleic acid construct may be in any suitable form, such as in the form of a plasmid, phage, transposon, cosmid, chromosome, vector, etc., which is capable of replication when associated with the proper control elements and which can transfer gene sequences, contained within the construct, between cells.

Thus, the term vector includes cloning and expression vehicles, as well as viral vectors. In some embodiments, the nucleic acid construct is a vector. In some embodiments, the vector is a viral vector, and therefore the present invention provides a viral vector including a nucleic acid molecule, or the nucleic acid construct, which encode the CAR described above. In some embodiments, the vector is a DNA vector or mRNA vector.

In at least some embodiments, the present invention provides a nucleic acid molecule, or a nucleic acid construct, encoding the CAR described above, for use in preparing a genetically modified cell. Further, in at least some embodiments, the present invention provides a use of a nucleic acid molecule in the preparation of a vector for the transformation, transfection or transduction of a cell. Preferably, the cell is a T cell expressing one or more of CD3, CD4 or CD8. Cells suitable for genetic modification can be heterologous or autologous.

In some embodiments, the cell is used in a method, or in the preparation of

a medicament, for the prevention or treatment of cancer. Consequently, in some embodiments, the present invention provides the use of a vector in the preparation of a medicament for the prevention or treatment of cancer.

Methods are known in the art for the deliberate introduction (transfection/transduction) of exogenous genetic material, such as the nucleic acid construct, into eukaryotic cells. As will be understood the method best suited for introducing the nucleic acid construct into the desired host cell is dependent on many factors, such as the size of the nucleic acid construct, the type of host cell the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation (see Potter and Heller. “Transfection by Electroporation.” Curr. Prot. Mol. Bio., ed. Frederick M. Ausubel et al. 2003: Unit-9.3), sonoporations (Wang, M et al. Sci. Reps., 2018; 8: 3885), heat-shock or optical transfection; particle-based methods such as ‘gene gun’ delivery, magnetofection, or impalefection, lipid nanoparticles or viral transduction.

A variety of viral transduction techniques for mammalian cells are known in the art. Common viral vectors include lentivirus and retrovirus. An exemplary protocol is provided in Wang L et al., Proc. Natl. Acad. Sci., 2011; 108: E803-12. Alternative viral vectors include, HSV, Adenovirus and AAV (Howarth J et al. Cell. Bio. & Toxic., 2010, vol. 26, issue 1, pp 1-20).

In some embodiments, the present invention provides a lentivirus comprising a nucleic acid encoding a chimeric antigen receptor as described herein. Further, the present invention provides a use of a viral vector, preferably a retrovirus such as a lentivirus or a gamma retrovirus, in the preparation of a genetically modified cell or a medicament for the prevention or treatment of cancer or for the killing of a cell, expressing Lgr5 or aberrantly expressing Lgr5.

The transduction of cells can result in genomic integration of DNA encoding the CAR described above. Alternatively, the DNA can be transiently expressed within the transduced cell. Each of these has positives and negatives. Genomic integrated DNA is stably expressed and replicated to progeny cells during cell replication. This ensures a robust immune response and a significant increase in CAR-expressing T cells in vivo.

Alternatively, transient transduction (often achieved by transducing with

mRNA) leads to temporary CAR expression in cells. This normally leads to a much lower response but provides more control to the practitioner to increase or decrease the “dosage” as needed.

As described above, in some embodiments, the invention provides the use of a DNA vector, or recombinant DNA, in the preparation of a viral vector for the genetic 5 transduction of a cell. The cell can be any cell, however suitable examples are provided below.

The nucleic acid construct will be selected depending on the desired method of transfection/transduction. In some embodiments, the nucleic acid construct is a viral vector, and the method for introducing the nucleic acid construct into a host cell is viral 10 transduction. Methods are known in the art for utilising viral transduction to elicit expression of a CAR in a PBMC such as a T cell (Parker, LL. et al. Hum Gene Ther. 2000; 11: 2377-87) and more generally utilising retroviral systems for transduction of mammalian cells (Cepko, C. and Pear, W. Curr Protoc Mol Biol. 2001, unit 9.9). In some embodiments, the nucleic acid construct is a plasmid, a cosmid, an artificial 15 chromosome or the like, and can be transfected into the cell by any suitable method known in the art.

Techniques are known in the art for selection/isolation of cell subsets. These include Fluorescent Activated Cell Sorting (Basu S. et al. J. Vis. Exp. 2010; 41: 1546), techniques utilising antibodies immobilised on a substrate, such as magnetic cell isolation (MACS®) device to immunomagnetically select cells expressing the desired markers (Zola H. et al. Blood, 2005; 106(9): 3123-6), or use of microfluidic chips. A series of cell markers can be used to isolate cells of the immune system including (but not limited to), BCR, CCR10, CD1a, CD1b, CD1c, CD1d, CD3, CD4, CDS, CD7, CD8, CD10, CD11b, CD11c, CD13, CD16, CD19, CD21, CD23, CD25, CD27, CD31, CD32, CD33, CD34, CD38, CD39, CD40, CD43, CD45, CD45RA, CD45RO, CD48, CD49d,

CD49f, CD51, CD56, CD57, CD62, CD62L, CD68, CD69, CD62, CD62L, CD66b, CD68, CD69, CD73, CD78, CD79a, CD79b, CD80, CD81, CD83, CD84, CD85g, CD86, CD94, CD103 CD106, CD115, CD117, CD122, CD123, CD126, CD127, CD130, CD138, CD140a, CD140b, CD141, CD152, CD159a, CD160, CD161, CD163, CD165, 30 CD169, CD177, CD178, CD183, CD185, CD192, CD193, CD194, CD195, CD196,

CD198, CD200, CD200R, CD203c, CD205, CD206, CD207, CD209, CD212, CD217, CD218 alpha, CD229, CD244, CD268, CD278, CD279, CD282, CD284, CD289,

CD294, CD303, CD304, CD314, CD319, CD324, CD335, CD336, CXCR3, Dectin-1, Tc epsilor R1 alpha, Flt3, Granzyme A, Granzyme B, IL-9, IL-13aphal, IL-21R, iNOS, KLRG1, MARCO, MHC class II, RAG, ROR Gamma T, Singlec-8, ST2, TCR alpha/beta, TCR gamma/delta, TLR4, TLR7, VEGF, ZAP70

Of particular note are the T cell markers CCR10, CD1a, CD1c, CD1d, CD2,

CD3, CD4, CDS, CD7, CD8, CD9, CD10, CD11 b, CD11c, CD13, CD16, CD23, CD25, CD27, CD31, CD34, CD38, CD39, CD43, CD45, CD45RA, CD45RO, CD48, CD49d, CD56, CD62, CD62L, CD68, CD69, CD73, CD79a, CD80, CD81, CD83, CD84, CD86, CD94, CD103, CD122, CD126, CD127, CD130, CD140a, CD140b, CD152, CD159a, CD160, CD161, CD165, CD178, CD183, CD185, CD192, CD193, CD194, CD195,

CD196, CD198, CD200, CD200R, CD212, CD217, CD218 alpha, CD229, CD244, CD278, CD279, CD294, CD304, CD314, CXCR3, Flt3, Granzyme A, Granzyme B, IL- 9, IL-13alphal, IL-21R, KLRG1, MHC class II, RAG, ROR gamma T, ST2, TCR alpha/beta, TCR gamma/delta, ZAP70. Particularly preferred cell markers for T cell selection include TCRgamma, TCR delta, CD3, CD4 and CD8.

Isolated cells can then be cultured to modify cell activity, expanded or activated. Techniques are known in the art for expanding and activating cells (Wang X. and Rivière I. Mol. Thera. Oncolytics. 2016; 3: 16015). These include; using anti- CD3/CD28 microbeads (Miltenyi Biotec or Thermofisher Scientific - as per manufacturer's instructions), or other forms of immobilised CD3/CD28 activating antibodies. Activated/genetically modified cells can then be expanded in vitro in the presence of cytokines (such as with IL-2, IL-12, IL-15 or IL-17) and then cryopreserved. An overview of methods for expanding CAR T cells is provided in Wang and Riviera ibid).

The present invention further provides a genetically modified cell including

the chimeric antigen receptor, nucleic acid molecule, or nucleic acid construct as described above. In some embodiments, the genetically modified cell includes a genomically integrated form of the nucleic acid molecule or construct. In some embodiments, the genetically modified cell is a leukocyte. In some embodiments, the genetically modified cell is a Peripheral Blood Mononuclear Cell (PBMC). In some embodiments, the genetically modified cell is a myeloid cell. In some embodiments, the genetically modified cell is a monocyte. In some embodiments, the genetically modified cell is a macrophage. In some embodiments, the genetically modified cell is a lymphocyte. In some embodiments, the genetically modified cell is a T cell. In some embodiments, the genetically modified cell is an alpha beta (a13) T cell. In some embodiments, the genetically modified cell is a gamma delta (0) T cell. In some embodiments, the genetically modified cell is a CD3+T cell (such as a naive CD3+T cells or a memory CD3+T cell). In some embodiments, the T cell is a CD4+T cell (such as a naive CD4+T cells or a memory CD4+T cell). In some embodiments, the T cell is a CD8+T cell (such as a naive CD8+T cells or a memory CD8+T cell). In some embodiments, the genetically modified cell is a natural killer cell. In some embodiments, the genetically modified cell is a natural killer T cell.

Use of Chimeric Antigen Receptor Expressing Cells.

Genetic modified cells, such as the CAR T cells described herein, can be used to target cells expressing Lgr5, and (depending on the cell type) may assist in, or lead to, killing of the cell expressing Lgr5 or aberrantly expressing Lgr5. In some embodiments, the present invention provides a method of killing a cell expressing Lgr5, or aberrantly expressing Lgr5, the method including exposing the cell expressing Lgr5 to a genetically modified cell having a chimeric antigen receptor (such as the CAR T cells described above), wherein the chimeric antigen receptor is directed against Lgr5.

In some embodiments, the genetically modified cell is autologous to the cell expressing Lgr5. In some embodiments, the cell expressing, or aberrantly expressing,

Lgr5 is within the body of a subject. In some embodiments, the cell expressing, or aberrantly expressing, Lgr5 is a cancer cell. In some embodiments the subject is a human.

Therefore, the present invention provides a use of a genetically modified cell as described above for preventing or treating cancer. Accordingly, the present invention provides a method of preventing or treating a patient having cancer, the method including exposing the patient to a cell expressing a chimeric antigen receptor, wherein the chimeric antigen receptor targets Lgr5. Preferably, the patient is administered a cell or genetically modified cell expressing the chimeric antigen receptor.

Furthermore, the invention provides a method of killing a cell expressing

Lgr5, the method comprising contacting the cell expressing Lgr5 with a cell expressing a CAR as describe above. In some embodiments, the cells expressing, or aberrantly expressing, Lgr5 is a cancer cell.

In some embodiments, the method of killing a cell expressing, or aberrantly expressing, Lgr5 and the method of preventing or treating a patient further include analysing the surface expression of Lgr5 on a target cell or a cancer cell. In some embodiments, the surface expression of Lgr5 on the target cells and cancer cells is compared to comparable non-cancerous cells. These comparable cells can be autologous or heterologous. The method may be performed in vitro or in vivo.

In some embodiments, the analysis of the surface expression of Lgr5 is performed prior to exposing the target cell, or cancer cells, to a cell or genetically modified cell expressing a chimeric antigen receptor.

In some embodiments, the method of killing target cell is performed when the target cells are cancer cells and aberrantly express Lgr5 when compared to non- cancerous cells. In some embodiments of the method of treating cancer in a patient, the cell expressing a CAR is administered if cancer cells overexpress Lgr5 compared to comparable non-cancerous cells.

In some embodiments, the method of preventing or treating cancer in a patient includes identifying the presence of cancer stem cells in a patient prior to exposing the patient to a cell or genetically modified cell expressing a chimeric antigen receptor.

Markers for cancer stem cells include (but are not limited to): ABCBS, ALDH1A1, CD200, CD133, CD44, CD34, CD24, EpCAM and Lgr5

Use of markers to identify cancer stem cells in various cancers types are known in the art. Examples of which includes: breast cancer (Ferro de Beca F. et al., J Clin Pathol, 2013; 66(3): 187-91), colorectal cancer (Munro M. et al., J Clin Pathol., 2018; 71(2): 110 and Cherciu I. et al. Curr Health Sci J., 2014; 40(3): 153-161), lymphoma (Kim S. et al., Korean J Hematol., 2011; 46(4): 211-213 and Song S. et al., J Cancer., 2020; 11(1): 142-152), gastrointestinal cancer (Ahmad R. et al. Biochem Pharmacol., 2016; 5(2): 202), lung cancer (Templeton A. et al., Stem Cell lnvestig., 2014; 1(9) and Huang Z. et al., J Cancer., 2017; 8(16): 3190-3197), glioblastoma

(Lathia J. et al., Genes Dev. 2015; 29(12): 1203-17), pancreatic cancer (Gzil A. et al., Mol Biol Rep., 2019; 46: 6629-45), and liver cancer (Sun J. et al., World J Gastroenterol., 2016; 7(22): 3547-57).

In some embodiments, the cancer is a solid cancer. In some embodiments, the cancer is selected from the group consisting of: bladder cancer, brain cancer, breast cancer, cervical cancer, colon cancer, endometrial cancer, epithelial cancers, oesophageal cancer, lung cancer, mouth cancer, ovarian cancer, kidney cancer, liver cancer, leukaemia, lymphoma, myeloma, pancreatic cancer, prostate cancer, rectal cancer, skin cancer, stomach cancer, testicular cancer, thyroid cancer and tongue cancer.

In some embodiments, the cancer is selected from the group consisting of: breast, pancreatic, prostate, colon, colorectal, lung (NSCLC), lymphoma, ovarian, gastrointestinal or B-cell lymphoma. In some embodiments, the cancer is selected from the group consisting of: colorectal, colon, B-cell lymphoma, ovarian cancer or a gastrointestinal cancer. In some embodiments, the cancer is a metastatic cancer. In some embodiments, the cancer is stage III cancer or is stage IV cancer

In some embodiments, the cancer is a hematological cancer. In some embodiments the cancer is selected from the group consisting of: leukemia, lymphoma and/or myeloma. In some embodiments, the leukemia is acute lymphocytic leukemia (ALL), acute myelogenous leukemia (AML), chronic lymphocytic leukemia (CLL) or chronic myelogenous leukemia (CML). In some embodiments, the lymphoma is Hodgkin lymphoma or is non-Hodgkin lymphoma. In some embodiments, the myeloma is IgG, IgA, IgM, IgD or IgE. In some embodiments, the myeloma is light chain myeloma, or non-secretory myeloma.

In some embodiments, the CAR according to the present invention, when

expressed in a cytotoxic T lymphocyte (CTL), induces cytotoxicity in vitro against target cells expressing, or aberrantly expressing, Lgr5 of at least 20%, at least 30%, at least 40% or at least 50%, or at least 60%, or at least 70%, or at least 80%, or at least 90%, or at least 95%, at a ratio of CAR Transduced CTL: target cells of 30:1 or greater, 10:1 or greater, 3:1 or greater or 1:1 or greater. Primarily CTLs express CD8 (i.e. CD8+) however a subset of CD4 expressing cells have been demonstrated to possess cytotoxic characteristics (Takeuchi, A. and Saito, T, Front. Immunol. 2017; 8: art.194). Therefore, in some embodiments the CTL is CD8+. In some embodiments the CTL is CD4+.

In some embodiments, the chimeric antigen receptor according to the 5 present invention, when expressed in a CD4+T-helper cell, increases IL-2, TNF alpha and/or IFN gamma production when co-cultured with a target cell expressing, or aberrantly expressing, Lgr5. In some embodiments, the increase is a statistically significant increase. In some embodiments the statistically significant increase is to a P-value of 0.05, 0.01 or 0.001.

10 The present invention further provides the use of a chimeric antigen receptor

as described herein, when expressed in an immune cell, for treating a cancer. Appropriate immune cells include the genetically modified cells disclosed above

The present invention also provides a pharmaceutical composition including a genetically modified cell including a chimeric antigen receptor, a nucleic acid molecule 15 or a nucleic acid construct as described above and one or more of a pharmaceutically acceptable carrier, excipient or diluent.

As used herein, “carrier”, “excipient” or “diluent” includes (but is not limited to) any solvent, dispersion medium, vehicle, coating, diluent, antibacterial, and/or antifungal agent, isotonic agent, absorption delaying agent, buffer, suspension, colloid, 20 or the like. The use of such media and/or agents for pharmaceutical active substances is well known in the art. Any conventional media or agent which is incompatible with genetically modified cells, is contemplated for use in a pharmaceutical composition. Supplementary active ingredients also can be incorporated into the compositions. “Pharmaceutically acceptable” means any material that is not biologically undesirable, 25 or undesirably reactive or toxic, and may be administered to an individual along with genetically modified cells expressing a chimeric antigen receptor without causing any undesirable biological effects or interacting in a deleterious manner with any of the other components of the pharmaceutical composition (particularly the genetically modified cells) in which it is contained. In some embodiments, the pharmaceutical composition 30 includes a further active ingredient that works simultaneously, cooperatively or synergistically. In some embodiments, the pharmaceutical composition includes a cytokine.

The pharmaceutical composition may be formulated in a variety of forms adapted to a preferred route of administration. Thus, a composition can be administered via known routes including, for example, parenteral (e.g., intradermal, transcutaneous, subcutaneous, intramuscular, intravenous, intraperitoneal, etc.). A pharmaceutical composition also can be administered via a sustained or delayed release. The pharmaceutical composition that includes genetically modified cells expressing a chimeric antigen receptor may be provided in any suitable form, including, but not limited to, a solution, a suspension, an emulsion, a spray, an aerosol, or any form of mixture.

In some embodiments, the pharmaceutical composition may be administered from about once to about five times per week. In some embodiments the pharmaceutical composition is administered once. In some embodiments, the pharmaceutical composition is administered twice. In some embodiments, the pharmaceutical composition is administered three times. In some embodiments, the pharmaceutical composition is administered four times.

In some embodiments, the pharmaceutical composition includes at least 5 x 8 cells. In some embodiments, the pharmaceutical composition includes at least 3 x 10 8 cells. In some embodiments, the pharmaceutical composition includes at least 2.5 x 10 8 cells. In some embodiments, the pharmaceutical composition includes at least 1 x 10 8 cells. In some embodiments, the pharmaceutical composition includes at least 5 x 10 7 cells. In some embodiments, the pharmaceutical composition includes at least 2.5 x 10 7 cells. In some embodiments, the pharmaceutical composition includes at least 1 x 10 7 cells. In some embodiments, the pharmaceutical composition includes at least x 10 6 cells. In some embodiments, the pharmaceutical composition includes at least 2.5 x 10 6 cells. In some embodiments, the pharmaceutical composition includes at least 1 x 10 6 cells.

In some embodiments, the pharmaceutical composition is administered to provide at least 5 x 10 8 cells. In some embodiments, the pharmaceutical composition is administered to provide at least 3 x 10 8 cells. In some embodiments, the pharmaceutical composition is administered to provide at least 2.5 x 10 8 cells. In some embodiments, the pharmaceutical composition is administered to provide at least 1 x 8 cells. In some embodiments, the pharmaceutical composition is administered to provide at least 5 x 10 7 cells. In some embodiments, the pharmaceutical composition is administered to provide at least 2.5 x 10 7 cells. In some embodiments, the pharmaceutical composition is administered to provide at least 1 x 10 7 cells. In some embodiments, the pharmaceutical composition is administered to provide at least 5 x 6 cells. In some embodiments, the pharmaceutical composition is administered to provide at least 2.5 x 10 6 cells. In some embodiments, the pharmaceutical composition is administered to provide at least 1 x 10 6 cells.

Generally, the pharmaceutical composition is administered to a subject in an amount, and in a dosing regimen, effective to reduce, limit the progression of, ameliorate, or resolve, to any extent, the symptoms or clinical signs of cancer. As used herein, “ameliorate” refers to any reduction in the extent, severity, frequency, and/or likelihood of a symptom or clinical signs of cancer. “Symptom” refers to any subjective evidence of disease or of a patient's condition. “Sign” or “clinical sign” refers to an objective physical finding relating to a particular condition. In the context of cancer, the composition is administered to a subject in an amount, and in a dosing regimen effective to limit the growth of one or more tumours, reduce the size, volume or weight of one or more tumours, reduce the rate of metastasis of the cancer or number of metastases, reduce the proliferation of cancer cells, or extend the life expectancy of a subject.

Definitions and Qualifications

The nucleotide and polypeptide sequences referred to herein are represented by a sequence identifier number (SEQ ID No.). A summary of the sequence identifiers is provided in Table 4. A sequence listing is also provided as part of the specification.

TABLE 4
Sequences Descriptions

| | | Sequence |
|---|---|---|
| | | Type Amino |
| Sequence | | Acid (A.A) |
| Identifier | Sequence | or DNA |
| 1 | LGR5 Amino Acid Sequence | A.A. |
| 2 | IgG1 hinge amino acid sequence | A.A. |
| 3 | IgG2 hinge amino acid sequence | A.A. |
| 4 | IgG3 hinge amino acid sequence | A.A. |
| 5 | IgG4 hinge amino acid sequence | A.A. |
| 6 | IgG4 hinge (mutated) amino acid sequence | A.A. |
| 7 | IgA1 hinge amino acid sequence | A.A. |
| 8 | IgD hinge amino acid sequence | A.A. |
| 9 | IgM common heavy (CH) 2 amino acid | A.A. |
| | sequence | |
| 10 | IgE CH2 amino acid sequence | A.A. |
| 11 | CD28 hinge amino acid sequence | A.A. |
| 12 | CD8 hinge (1) amino acid sequence | A.A. |
| 13 | CD8 hinge (2) amino acid sequence | A.A. |
| 14 | CD8b hinge (1) amino acid sequence | A.A. |
| 15 | CD8b hinge (2) amino acid sequence | A.A. |
| 16 | CD7 hinge amino acid sequence | A.A. |
| 17 | CD4 hinge amino acid sequence | A.A. |
| 18 | PolyG/S amino acid sequence | A.A. |
| 19 | IgG1 heavy domain amino acid sequence | A.A. |
| 20 | IgG2 heavy domains amino acid sequence | A.A. |
| 21 | IgG3 heavy domains amino acid sequence | A.A. |
| 22 | IgG4 heavy domains amino acid sequence | A.A. |
| 23 | IgG1 CH2 amino acid sequence | A.A. |
| 24 | IgG1 CH3 amino acid sequence | A.A. |
| 25 | IgG2 CH2 amino acid sequence | A.A. |
| 26 | IgG2 CH3 amino acid sequence | A.A. |
| 27 | IgG3 CH2 amino acid sequence | A.A. |
| 28 | IgG3 CH3 amino acid sequence | A.A. |
| 29 | IgG4 CH2 amino acid sequence | A.A. |
| 30 | IgG4 CH3 amino acid sequence | A.A. |
| 31 | CD3 zeta amino acid sequence | A.A. |
| 32 | CD3 epsilon amino acid sequence | A.A. |
| 33 | CD3 gamma amino acid sequence | A.A. |
| 34 | CD3 delta amino acid sequence | A.A. |
| 35 | FCεR1 amino acid sequence | A.A. |
| 36 | FCγR1 amino acid sequence | A.A. |
| 37 | VH CDR1 | A.A. |
| 38 | VH CDR2 | A.A. |
| 39 | VH CDR3 | A.A. |
| 40 | VL CDR1 | A.A. |
| 41 | VL CDR2 | A.A. |
| 42 | VL CDR3 | A.A. |
| 43 | VH CDR1 | DNA |
| 44 | VH CDR2 | DNA |
| 45 | VH CDR3 | DNA |
| 46 | VL CDR1 | DNA |
| 47 | VL CDR2 | DNA |
| 48 | VL CDR3 | DNA |
| 49 | Variable Light Chain | A.A. |
| 50 | Variable Heavy Chain | A.A. |
| 51 | Variable Light Chain 30xx | DNA |
| 52 | Variable Heavy Chain 30xx | DNA |
| 53 | Binding Domain CNA30xx-Variable Light- | A.A. |
| | Variable Heavy | |
| 54 | Binding Domain CNA31xx-Variable Heavy- | A.A. |
| | Variable Light | |
| 55 | IgG4 mutant hinge linker | A.A. |
| 56 | hinge-CH3 linker | A.A. |
| 57 | hinge-CH2—CH3 linker | A.A. |
| 58 | CD3 Zeta signalling domain | A.A. |
| 59 | 4-1BB Signalling Domain | A.A. |
| 60 | CNA3002 | A.A. |
| 61 | CNA3003 | A.A. |
| 62 | CNA3004 | A.A. |
| 63 | CNA3102 | A.A. |
| 64 | CNA3103 | A.A. |
| 65 | CNA3104 | A.A. |
| 66 | CNA3002 | DNA |
| 67 | CNA3003 | DNA |
| 68 | CNA3004 | DNA |
| 69 | CNA3102 | DNA |
| 70 | CNA3103 | DNA |
| 71 | CNA3104 | DNA |
| 72 | CD28 trans membrane domain | A.A. |
| 73 | ScFv CNA30xx-Variable Light-Variable Heavy | DNA |
| 74 | ScFv CNA31xx-Variable Heavy-Variable Light | DNA |
| 75 | CNA30xx-IgG4 mutant hinge linker | DNA |
| 76 | CNA30xx-hinge-CH3 linker | DNA |
| 77 | CNA30xx-hinge-CH2—CH3 linker | DNA |
| 78 | CD3 Zeta signalling domain | DNA |
| 79 | T2A self-cleavage site | A.A. |
| 80 | T2A self-cleavage site | DNA |
| 81 | EGFRt | A.A. |
| 82 | EGFRt | DNA |
| 83 | IgG4 CH3 region | A.A. |
| 84 | IgG4 CH2 region | A.A. |
| 85 | light chain leader sequence | A.A. |
| 86 | light chain leader sequence | DNA |
| 87 | heavy chain leader sequence | A.A. |
| 88 | heavy chain leader sequence | DNA |
| 89 | CNA31xx-IgG4 mutant hinge linker | DNA |
| 90 | CNA31xx-hinge-CH3 linker | DNA |
| 91 | CNA31xx-hinge-CH2—CH3 linker | DNA |
| 92 | xx02/03-CD28 trans membrane domain | DNA |
| 93 | xx04-CD28 trans membrane domain | DNA |
| 94 | 4-1BB Signalling Domain | DNA |
| 95 | Ab 18G7.1 heavy chain CDR1 | A.A. |
| 96 | Ab 18G7H6A1 heavy chain CDR3 | A.A. |
| 97 | Ab 18G7.1 light chain CDR3 | A.A. |
| 98 | Scfv fusion domain | A.A. |
| 99 | Ab 18G7.1 heavy chain | A.A. |
| 100 | Ab 18G7.1 light chain | A.A. |
| 101 | 18G7H6A1 heavy chain | A.A. |
| 102 | 18G7H6A1 light chain | A.A. |
| 103 | Variable Light Chain 31xx | DNA |
| 104 | Variable Heavy Chain 31xx | DNA |

As discussed herein, the sequences listed above include functional variants which may have modifications and mutations in the sequence. As would be understood to those skilled in the art a “functional variant” still maintains a portion of, or all, of the function of the original protein or nucleic acid of the sequence. As such, the functional variant may, for example, have one or more amino acid insertions, deletions or substitutions relative to one of SEQ ID Nos. provided above. Or, in the case of a nucleic acid, a function variant may include one or more synonymous mutation(s) thereby still encoding the same amino acid sequence, or may include one or more non-synonymous mutation(s) so long as the encoded protein is a functional variant of the originally encoded protein.

By way of example, a functional variant of an antibody or a binding portion of an antibody would be any variant that provides same, or similar specificity. With regard to an antibody the function of the antibody must be considered in its context of use. For example, the functionality of a binding portion of an antibody is its ability to recognise an epitope. However, any antibody in its entirety may also functions to induce an immune response such as activating complement or activating effector cells.

In some embodiments, a functional variant, or variant, may comprise at least 50% amino acid sequence identity, at least 55% amino acid sequence identity, at least 60% amino acid sequence identity, at least 65% amino acid sequence identity, at least 70% amino acid sequence identity, at least 75% amino acid sequence identity, at least 80% amino acid sequence identity, at least 85% amino acid sequence identity, at least 90% amino acid sequence identity, at least 91% amino acid sequence identity, at least 92% amino acid sequence identity, at least 93% amino acid sequence identity, at least 94% amino acid sequence identity, at least 95% amino acid sequence identity, at least 96% amino acid sequence identity, at least 97% amino acid sequence identity, at least 98% amino acid sequence identity, at least 99% amino acid sequence identity, or at least 99.1%, 99.2%, 99.3%, 99.4%, 99.5%, 99.6%, 99.7%, 99.8%, or 99.9% amino acid sequence identity to any one of SEQ ID Nos. listed and recited herein. In some embodiments, the function variant maintains 50%, 60%, 70%, 80%, 90%, 95%, 98%, 99% or 99.9% of the function of the original peptide/protein.

As used herein “aberrantly expressing” relates to any expression that deviates from normal expression for a comparable cell. In some embodiments, aberrantly expressing mean expressing a mutated form of Lgr5 or a dysfunctional or non-functional form of Lgr5. In some embodiments, aberrantly expressing means over- expressing of Lgr5 compared to a normal cell. In some embodiments over-expression means at least 10% elevation in expression, or at least 20% elevation in expressing, or at least 30% elevation in expression, or at least 40% elevation in expression, or at least 50% elevation in expression, or at least 60% elevation in expression, or at least 70% elevation in expression, or at least 80% elevation in expression, or at least 90% elevation in expression, or at least 100% elevation in expression, or at least 125% elevation in expression, or at least 150% elevation in expression, or at least 175% elevation in expression, or at least 200% elevation in expression, or at least 250% elevation in expression, or at least 300% elevation in expression, or at least a 350% elevation in expression, or at least a 400% elevation in expression, or at least a 450% elevation in expression or at least a 500% elevation in expression when compared to a comparable normal cell.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be further understood that terminology such as “comprise”, or

variations such as “comprises” or “comprising” inherently include within their scope (without being limited to) versions of the invention that excludes other elements directly related to the inventions. Accordingly, terminology such as “consisting of” or “consisting essentially of” can be substituted for terminology such as “comprise”, “comprises” or “comprising” with the effect of limiting the scope of the invention to the specifically recited elements. Notably, where it is explicitly intended for the invention to be considered in an exhaustive manner, such limitations should be considered to relate only to the inventive concept disclosed herein and other features can be added which fall outside of the scope of the inventive concept. Such features or elements may include, but are not limited to, excipients, formulations, additives, diluents, packaging, adjuvants and collocated features which are not to be excluded by terminology such as “consisting of” or “consisting essentially of”.

When comparing nucleic acid sequences, the sequences should be compared over a comparison window which is determined by the length of the nucleic acid or is otherwise specified. For example, a comparison window of at least 20 residues, at least 50 residues, at least 75 residues, at least 100 residues, at least 200 residues, at least 300 residues, at least residues, at least 500 residues, at least 600 residues, or over the full length of any one of the sequences listed in Table 4. The comparison window may comprise additions or deletions of about 20%, about 18%, about 16%, about 14% about 12%, about 9%, about 8%, about 6%, about 4% or about 2% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al., Nucl. Acids Res. 1997; 25: 3389-3402. Global alignment programs may also be used to align similar sequences of roughly equal size. Examples of global alignment programs include NEEDLE (available at www.ebi.ac.uk/Tools/psa/emboss_needle/) which is part of the EMBOSS package

(Rice P et al., Trends Genet., 2000; 16: 276-277), and the GGSEARCH program (available at fasta. bioch.virginia.edu/fasta_www2/fasta_www.cgi?rm=compare&pgm =gnw) which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al Eds. (“Current Protocols in Molecular Biology” John Wiley & Sons Inc, Chapter 19, 2003).

The term “substitution” refers to replacement of an amino acid at a particular position in a parent peptide or protein sequence with another amino acid. A substitution can be made to change an amino acid in the resulting protein in a non-conservative manner (e.g., by changing the amino acid belonging to a grouping of amino acids having a particular size or characteristic to an amino acid belonging to another grouping; e.g. substituting a hydrophilic amino acid with a hydrophobic amino acid) or in a conservative manner (e.g., by changing the amino acid belonging to a grouping of amino acids having a particular size or characteristic to an amino acid belonging to the same grouping; e.g. substituting a hydrophilic amino acid with a hydrophilic amino acid). Such a conservative change generally leads to a reduction in conformational and functional changes in the modified peptide/protein. The following are examples of various groupings of amino acids: 1) Amino acids with nonpolar R groups: Alanine,

Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

A person skilled in the art will recognise that any amino acid can be substituted with a chemically (functionally) similar amino acid and retain function of the polypeptide. Such conservative amino acid substitutions are well known in the art. The following groups in Table 5 and 6 provide some conservative amino acids.

TABLE 5
Exemplary amino acid conservative substitutions

| Original | Exemplary |
|---|---|
| Residue | Substitutions |
| Ala (A) | Val (V), Leu (L), Ile (I), Gly (G) |
| Arg (R) | Lys (K) |
| Asn (N) | Gln (Q), His (H) |
| Asp (D) | Glu (E) |
| Cys (C) | Ser (S) |
| Gln (Q) | Asn (N), His (H) |
| Glu (E) | Asp (D) |
| Gly (G) | Pro (P), Ala (A) |
| His (H) | Asn (N), Gln (Q) |
| Ile (I) | Leu (L), Val (V), Ala (A) |
| Leu (L) | Ile (I), Val (V), Met (M), Ala (A), Phe (F) |
| Lys (K) | Arg (R) |
| Met (M) | Leu (L), Phe (F) |
| Phe (F) | Leu (L), Val (V), Alal (A) |
| Pro (P) | Gly (G) |
| Ser (S) | Thr (T) |
| Thr (T) | Ser (S) |
| Trp (W) | Tyr (Y) |
| Tyr (Y) | Trp (W), Phe (F) |
| Val (V) | Ile (I), Leu (L), Met (M), Phe (F), Ala (A) |

TABLE 6
Conservative amino acid groups

| Family | Amino Acids |
|---|---|
| Aliphatic | Val (V), Leu (L), Ile (I), Gly (G) |
| Aromatic | Phe (F), Tyr (Y), Trp (W) |
| Hydroxyl or containing | Ser (S), Cys (C), Sec (U), Thr (T), Met (M) |
| sulfur/selenium | |
| Cyclic | Pro (P) |
| Basic | His (H), Lys (K), Arg (R) |
| Acidic or their amides | Asp (D), Glu (E), Asp (D), Gln (Q) |

The term “insertion” refers to addition of amino acids within the interior of the sequence. “Addition” refers to addition of amino acids to the terminal ends of the sequence. “Deletion” refers to removal of amino acids from the sequence.

As will be understood the term “modification” or “mutation” includes any

addition, deletion, insertion or substitution to an amino acids sequence, or a nucleic acid sequence.

Reference is made to standard textbooks of molecular biology that contain methods for carrying out basic techniques encompassed by the present invention. See, for example, Green MR and Sambrook J, Molecular Cloning: A Laboratory Manual (4th edition), Cold Spring Harbor Laboratory Press, 2012.

All methods described herein can be performed in any suitable order unless indicated otherwise herein or clearly contradicted by context. The use of any and all examples, or exemplary language (e.g., “such as”, “i.e.”) is intended merely to better illuminate the exemplified embodiments and does not pose a limitation on the scope of the claimed invention unless explicitly claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

It will be apparent to the person skilled in the art that while the invention is described herein in detail for the purposes of clarity and understanding, various modifications and alterations to the embodiments and methods described herein may be made without departing from the scope of the inventive concept disclosed in this specification.

Examples

The invention is further illustrated in the following examples. The examples

are for the purpose of describing particular embodiments only and are not intended to be limiting with respect to the above description

Example 1 — Design Preparation and Expression of an anti-Lgr5 Chimeric Antigen Receptor.

An exemplified protocol detailing the process of designing and expressing a

chimeric antigen receptor according to embodiments of the present invention is detailed as follows.

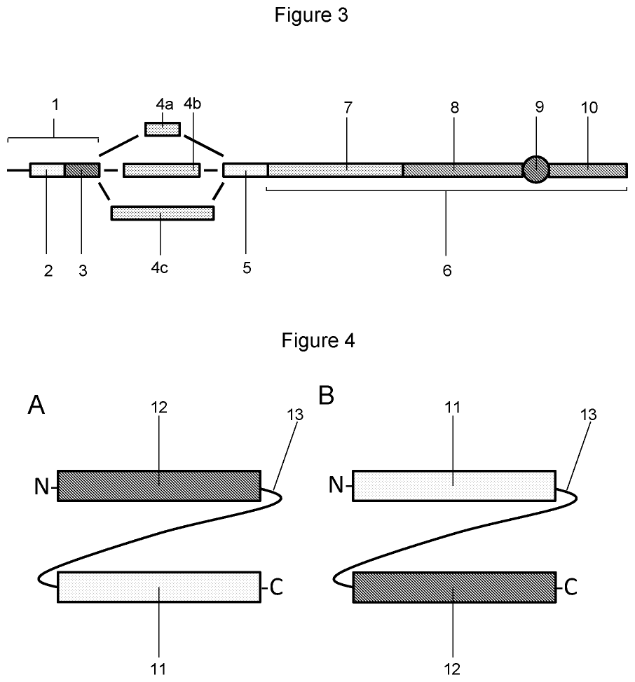

CAR constructs (collectively referred to as CNA CAR family constructs) were prepared as illustrated in FIG. 3 consisting of: an extracellular domain 1 comprising a leader sequence 2 which was either an antibody light chain leader sequence

(CNA30)o(— SEQ ID No. 85) or a heavy chain leader sequence (CNA31xx - SEQ ID No. 87), an antigen binding domain 3 directed against Lgr5 (CNA 30)o(— SEQ ID No. 53 and CNA31xx— SEQ ID No. 54), and one of three linker domains 4a-c (SEQ ID Nos. 56 and 57) linked to a CD28 transmembrane domain 5 (SEQ ID No. 72), and an endodomain 6 including the intracellular signalling domain which includes a signalling portion of 4-1BB 7 (SEQ ID No. 59), an intracellular signalling portion of the CD3-zeta chain 8 (SEQ ID No. 58), a T2A self-cleavage site 9 (SEQ ID No. 79), and a truncated form of the EGFR receptor (EGFRt) 10 (SEQ ID No. 81) that lacks an intracellular signalling domain. Consequently, the surface expression of EGFRt can be used as a proxy to measure transduction efficiency with the CAR construct.

As illustrated in FIG. 3, the extracellular domain 1 and the transmembrane domain 5 were linked by one of three linker domains being:

4a. A linker domain of 12 amino acids, comprising a mutated version of the

IgG4 hinge region (SEQ ID No. 55). These CARs are denoted with the series suffix (xx) number 02;

4b. A linker domain of 119 amino acids, comprising a mutated version of the

IgG4 hinge region and the IgG4 CH3 region (SEQ ID No. 83) to provide a linker domain having the amino acid sequence set forth in SEQ ID No. 56. CARs having this linker are denoted with the series suffix (xx) number 03; and 4c. A linker domain of 229 amino acids, comprising a mutated version of the

IgG4 hinge region, the IgG4 CH2 region (SEQ ID No. 84 and having L235D and N297Q mutations) to provide a linker domain having the amino acid sequence set forth in SEQ ID No. 57. CARs having this linker are denoted with the series suffix (xx) number 04 and the IgG4 CH3 region.

As illustrated in FIGS. 4A and 4B the antigen binding domain 3 of the ectodomain 1 consisted of one of two fusion proteins (denoted with the prefix CNA30xx and the prefix CNA31xx, wherein the suffix is one of the “xx” series number discussed above. Specifically, two single-chain variant fragment (scFv) fusion proteins were formed. These comprised the variable heavy (VH) chain 11 (SEQ ID No. 50) and the variable light (VL) chain 12 (SEQ ID No. 49) fused by an 18 amino acid fusion domain 13 (SEQ ID No. 98). FIG. 4A illustrates a first biding domain (CNA30xx —SEQ ID No. 53) wherein the C-terminus of the VL chain is connected to a N terminus of the VH chain by the fusion domain 13. A second binding domain (CNA31xx — SEQ ID No. 54) is illustrate in FIG. 4B wherein the C-terminus of the VH chain is connected to a N- terminus of the VL chain by the fusion domain 13. Two different orientations of the scFv were generated as the variable domain order of a CAR may influence the surface expression and activity of the CAR, as has been noted previously (Burns et al., Cancer Res: 2010, 70, 3027-3033).

Consequently, six different CARs were designed having one of three linker

regions and the two binding domains. These six CARs are summarised below:

CNA3002 — VL-VH-IgG4 hinge (short hinge — 12 a.a.): amino acid sequence — Seq ID No. 60. CNA3003 — VL-VH-IgG4 hinge-CH3 (medium hinge — 119 a.a.): amino acid sequence — Seq ID No. 61.

CNA3004 — VL-VH-IgG4 hinge-CH2-CH3 (long hinge — 229 a.a.): amino acid sequence — Seq ID No. 62. CNA3102 — VH-VL-IgG4 hinge (short hinge — 12 a.a.): amino acid sequence — Seq ID No. 63. CNA3103 — VH-VL-IgG4hinge-CH3 (medium hinge — 119 a.a.): amino acid sequence — Seq ID No. 64. CNA3104 — VH-VL-IgG4hinge-CH2-CH3 (long hinge — 229 a.a.): amino acid sequence — Seq ID No. 65.

Each of the six CAR constructs were human codon usage optimized. The respective sequences encoding each component is set out in Table 7, below. In total six CAR DNA coding sequences were synthesised (Seq ID Nos: 66, 67, 68, 69, 70 and 71, correspond to CNA3002, CNA3003, CNA3004, CNA3102, CNA3103 and CNA3104, respectively). These were synthesised as GeneBlockTM double stranded DNA fragments and cloned using NEB HiFi assembly reaction into digested epHIV-7.2 lentiviral DNA vector backbone to provide six vectors.

TABLE 7
Protein and coding DNA for CAR components

| CAR | Sequence | CNA prefix | | CNA |
|---|---|---|---|---|
| item | type | 30xx | 31xx | suffix |
| 2 | amino acid | SEQ ID No. 85 | SEQ ID No. 87 | xx02-xx04 |
| | DNA | SEQ ID No. 86 | SEQ ID No. 88 | xx02-xx04 |
| 3 | amino acid | SEQ ID No. 53 | SEQ ID No. 54 | xx02-xx04 |
| | DNA | SEQ ID No. 73 | SEQ ID No. 74 | |
| 4a | amino acid | SEQ ID No. 55 | | xx02 |
| | DNA | SEQ ID No. 75 | SEQ ID No. 89 | |
| 4b | amino acid | SEQ ID No. 56 | | xx03 |
| | DNA | SEQ ID No. 76 | SEQ ID No. 90 | |
| 4c | amino acid | SEQ ID No. 57 | | xx04 |
| | DNA | SEQ ID No. 77 | SEQ ID No. 91 | |
| 5 | amino acid | SEQ ID No. 72 | | xx02-xx03 |
| | DNA | SEQ ID No. 92 | | |
| | amino acid | SEQ ID No. 72 | | xx04 |
| | DNA | SEQ ID No. 93 | | |
| 7 | amino acid | SEQ ID No. 59 | | xx02-xx04 |
| | DNA | SEQ ID No. 94 | | |
| 8 | amino acid | SEQ ID No. 58 | | xx02-xx04 |
| | DNA | SEQ ID No. 78 | | |
| 9 | amino acid | SEQ ID No. 79 | | xx02-xx04 |
| | DNA | SEQ ID No. 80 | | |
| 10 | amino acid | SEQ ID No. 81 | | xx02-xx04 |
| | DNA | SEQ ID No. 82 | | |

Example 2 —Preparation of Lentiviral Vectors.

239 T cells were transiently transfected with vectors containing the CNA family of CARs to produce lentivirus.

5 293T cells were seeded in Opti-MEM GlutaMAX media supplemented with

5% serum and incubated at 37° C. with 5% CO2 to allow the cells to adhere to the flask.

Virus for the six different CNA CARs was produced by incubating in media CAR encoding DNA plasmids, viral packaging plasmids, Lipofectamine 3000 reagent (Invitrogen), P3000 enhancer reagent together with 293T cells.

Supernatants were collected and spun, to remove cellular debris. The supernatant was then filtered (0.45 pM) and virus was concentrated from the filtered supernatant by centrifugation and stored at -80 ° C.

Example 3 — Isolation and Transduction of T Cells.

CD3+T cells were isolated from whole blood using Rosettesep Human T

cell enrichment cocktail (StemCell), following the manufacturer's protocol.

Isolated CD3+T cells were stimulated with CD3/CD28 Dynabeads (Thermo Fischer Scientific) for 1 h after isolation (37° C., 5% CO2). The stimulated cells were subsequently transduced with one of the CNA3002, CNA3003, CNA3004, CNA3102, CNA3103 or CNA3104 CAR containing viruses. An untransduced sample was utilised as a control. The isolated cells were cultured in complete media containing polybrene at 37° C., 5% CO2 prior to the dynabeads being removed by magnetic separation. The cells were cultured further in complete media containing IL-21, IL-7 and IL-15. Cells were maintained by regular media changes and routinely split to maintain growth.

1x10 6 of each of the six CAR transduced T cells, as well as untransduced

control cells, were given a second stimulation using irradiated PBMC cells and CD3 antibody, in complete media including IL-21, IL-7 and IL-15. During this period, cells were feed and split as discussed above prior to being used for in vitro cytolysis assays (see below) or frozen for future use.

As indicated above, the CAR constructs include EGFRt connected to a T2A

self-cleavage peptide. Upon expression of the CAR constructs the EGFRt is cleaved from the mature chimeric antigen receptor polypeptide and is separately expressed on the cell surface. Consequently, surface expression of truncated EGFR (EGFRt) was used to measure transduction efficiency in T cells.

Transduction efficiency of the CD3 cells was determined on day 5 post-

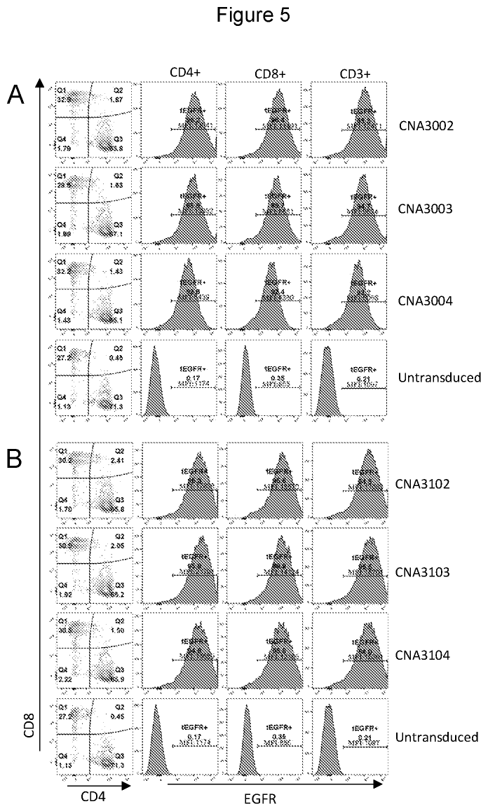

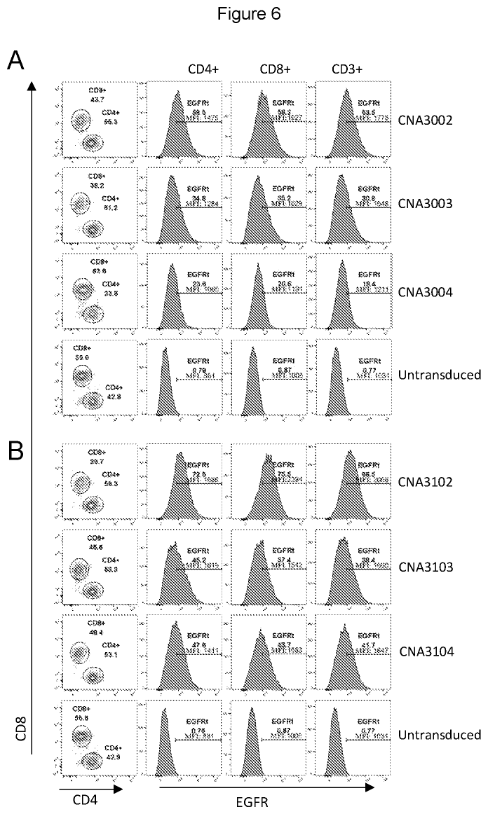

transduction (FIGS. 5A and 5B) and day 15 post-transduction (FIGS. 6A and 6B), by staining the transduced cells with anti-CD3 antibody, anti-CD4 antibody, anti-CD8 antibody and anti-EGFR antibody (eBiosciences) and analysing surface expression by flow cytometry.

As can be seen in FIGS. 5A and 5B, on day 5 post transduction all six groups of transduced CD3+T cells showed more than 90% EGFR expression indicating expression of the relevant CAR. Specifically, 95.3% of CD3+lymphocytes transduced with the CNA3002 CAR construct expressed EGFR, 94.7% of CD3+lymphocytes transduced with the CNA3003 CAR construct expressed EGFR, 92.2% of

CD3+lymphocytes transduced with the CNA3004 CAR construct expressed EGFR, 94.5% of CD3+lymphocytes transduced with the CNA3102 CAR construct expressed EGFR, 95.5% of CD3+lymphocytes transduced with the CNA3103 CAR construct expressed EGFR and 94.0% of CD3+lymphocytes transduced with the CNA3104 CAR construct expressed EGFR. By comparison only 0.21% of CD3+cells which were untransduced were stained with anti-EGFR antibody. However, by day 15 (FIGS. 6A and 6B) the EGFR expression had reduced.

Example 4 — CAR T cell Effector Function.

To assess the activity of the anti-Lgr5 CAR-expressing T cells, in vitro killing assays were performed.

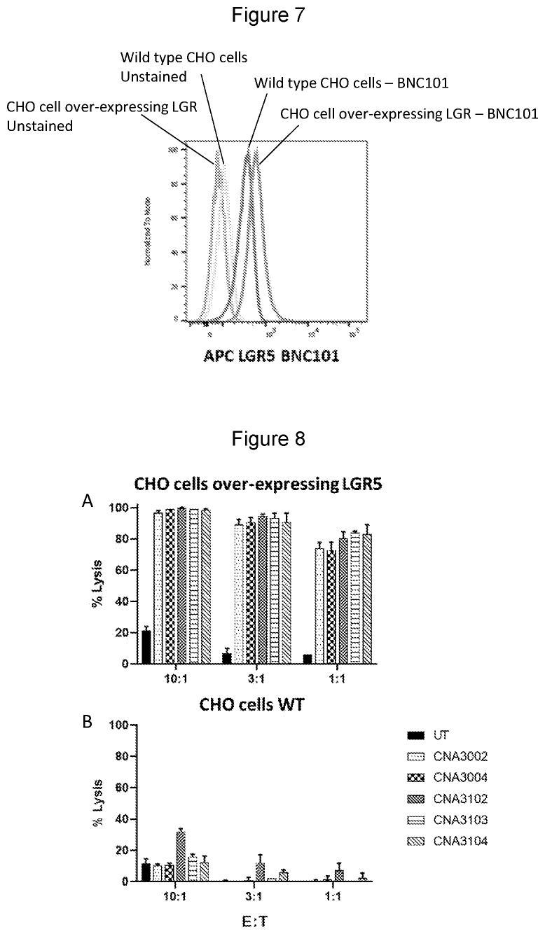

CHO cells which were modified to express FF luciferase (controls) and CHO cells which over-express Lgr5 and express FF luciferase were utilised as model target cells. FIG. 7 shows staining of CHO cells (wild-type and Lgr5 over-expressing) with the anti-Lgr5 antibody BNC101 conjugated to Alexa Fluor 647. Briefly, 2x10 5 CHO cells were washed with PBS and the BNC101 antibody was added to each tube at a final concentration of 400ug/u1 in a total volume of 50u1 of PBS. The CHO cells were incubated in the presence of the BNC101 antibody at 4° C. for 30 mins before being washed with PBS, resuspended and analysed with a flow cytometer.

As can be seen in FIG. 7, CHO cells overexpressing Lgr5 clearly showed elevated expression of Lgr5 relative to wild-type CHO cells indicating their suitability as target cells for the CNA CAR constructs.

CHO target cells described above were seeded at a concentration of 1x10 4 cells in 50 ul of media per well in round bottom 96-well plates. Each test was performed in triplicate and the results were averaged. Un-transduced T cells were used as controls.

T cells transduced with five of the six CAR constructs (CNA3002, CNA3004, CNA30102, CNA3103 and CNA3104) as well as untransduced (UT) T cells, were co- cultured with target cancer cell lines at T cell to target ratios of 10:1, 3:1 and 1:1 for a period of 18 hours at 37° C. with 5% CO2.

The Bright-Glo TM luciferase-based assay system was used to measure lysis

of target cell in the co-culture in accordance with the manufacturer's instructions.

As can be seen in FIGS. 8A all five tested anti-Lgr5 CAR constructs showed significant lysis (greater than 70%) of CHO target cell which over-expressed Lgr5, while untransduced T cell had less than 20% lysis. By comparison, wild type CHO cells were not significantly lysed by any of the CAR expressing T cells (FIG. 8B).

Example 5 — Anti-Lgr5 CAR T cell activity against tumour cell lines.

Having established the efficacy of the CAR expressing T cells against a model target cell, they were tested in vitro against the following cancer cell lines sourced from CellBank Australia: LoVo (metastatic colon epithelial cell cancer), LIM1215 (colorectal epithelial cell carcinoma), Raji (Burkitt's lymphoma), Namalwa

(Burkitt's lymphoma), OVCAR-3 (ovarian carcinoma), SHY-5Y-SY (neuroblastoma) and BE(2)-M17 (neuroblastoma).

The various cell lines (which stably express the luciferase reporter) were plated at 1x10 4 cells in 50 ul per well of a 96 well round bottom plate. Triplicate measures were performed for each ratio and T cell treatment tested. Untransduced T cells were used for control cells.

Each of the six CAR-T cell populations and untransduced T cells were co- cultured with target cancer cell lines at E:T ratios of 10:1, 3:1 and 1:1 for 18h. The BrightGlo luciferase-based assay system was used as described above to measure the cytolysis potential of the CD3 CAR-T cells against five different cancer cell lines.

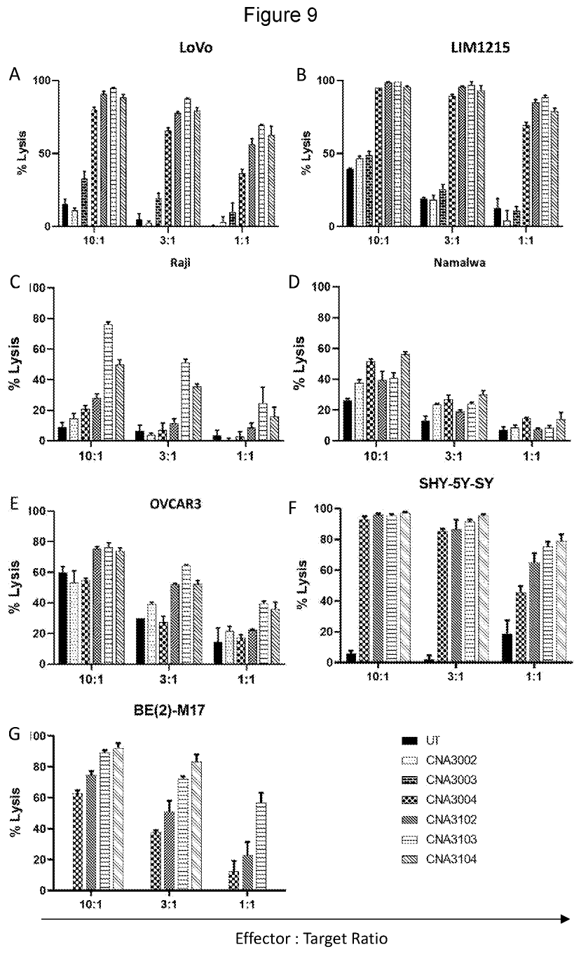

FIGS. 9A to 9G show efficacy of the anti-Lgr5 CAR expressing T cells against the seven cancer cell lines specified above.

As can be seen CNA3004 (VL-VH-long-hinge) and well as CNA3102 (VH- VL-short hinge), CNA3103 (VH-VL-medium hinge) and CNA3104 (VH-VL-long hinge) all showed effective lysis of LoVo, LIM1215 cells, Namalwa, SHY-5Y-SY and BE(2)- M17 cells. Further, CNA3102, CNA3103 and CNA3104 showed effective lysis of Raji cells particularly at the effector cell to target cell (E:T) ratio of 10 to 1 with CNA103 and CNA104 being most effective. Moreover, anti-Lgr5 CAR T cells effectively lysed OVCAR3 cells, with CNA3102, CNA3103 and CNA3104 being most effective at targeting OVCAR3.

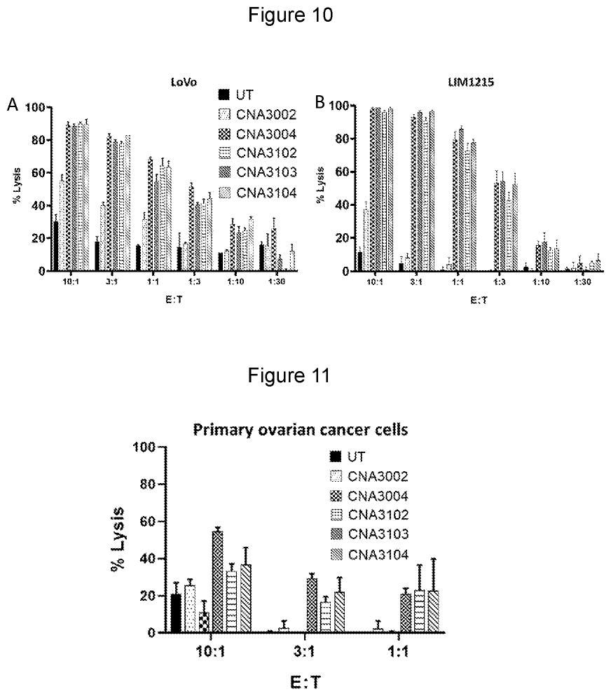

Having demonstrated that the anti-Lgr5 CAR T cells are effective at lysing LoVo and LIM1215 at effector to target cell ratios of 10:1, 3:1 and 1:1, further smaller ratios (1:3, 1:10 and 1:30) of effector to target cells were tested (FIG. 10).

As can be seen in FIGS. 10A and 10B, even at effector to target ratios of

1:10, anti-Lgr5 CAR T cells were able to effectively lyse cancer cells.

Example 6 — Anti-Lgr5 CAR T cell activity against primary tumour cells.

Having established that anti-Lgr5 CAR T cells can lyse CHO cells over- expressing Lgr5 and cancer cell lines, the efficacy of the anti-Lgr5 CAR T cells against primary ovary cancer cells was assessed.

Primary ovarian cancer cells (n=8) derived from ascites were collected from advanced stage serous ovarian cancer patients with informed patient consent and approval from the Royal Adelaide Hospital Human Ethics Committee. The culture method has been previously described (Carmon, K. S. et al., PNAS., 2011, 108, 11452- 7 and Ricciardelli, C. et al., Cancer Lett., 2018, 421, 51-58). Briefly, tumour cells from ascites were cultured in advanced RPMI-1640 medium (Life Technologies, Thermo Fisher Scientific, Waltham, MA, USA) supplemented with 2 mM GlutaMAX (Life Technologies), 10% FBS and PSF antibiotics. Primary ovarian cancer cell lines were stored in liquid nitrogen until use. These cells were modified to express FF luciferase.

Isolated primary ovarian cancer cells 1x10 4 were plated in 50p1 of media in

wells of a 96-well round bottom plate and co-incubated with various ratios of anti-Lgr5 CAR T cells. A BrightGlo luciferase cytolysis assay was performed as described above to assess lysis of target cells.

As can be seen in FIG. 11, anti-Lgr5 CAR T cells effectively lysed primary ovarian cancer cells at ratios as low as 1:1 (effector to target ratio).

Example 7 — Lrg5 blocking antibodies inhibit CAR T cell lysis

To confirm that the CNA CAR-T cells lyse target cells in an antigen-specific manner, the target epitope on Lgr5 was blocked using the antibody BNC101 (see above).

5 As demonstrated in FIGS. 9 and 10, LoVo colorectal cancer cells can be

lysed by CNA family CAR T cells. Accordingly, to assess antigen-specific lysis, LoVo cells were incubated with BNC101 antibody for 4 h, followed by washing with PBS and subsequently suspended in complete XVIVO media and plated at 1x10 4 cells/well in 96-well plates.

For negative controls an equal number of target cells were also incubated

for 4 h in PBS or complete XVIVO media prior to being washed with PBS and plated in complete XVIVO media as described above.

Four anti-Lgr5 CAR-T cells (CNA3004, CNA3102, CNA3103 and CNA3104) were co-incubated with the plated LoVo cell to produce effector to target cell ratios of 15 10:1, 3:1, 1:1, 1:3, 1:10 and 1:30. Following the addition of CAR T cells additional

BNC101 antibody (2mg/m1) was added to the treatment group (antibody pre-treated group). The plates were then incubated for 16 h at 37 ° C. with 5% CO2. At the completion of the 16 h incubation a BrightGlo luciferase cytolysis assay was performed (as described above) to assess the lysis of target cells.

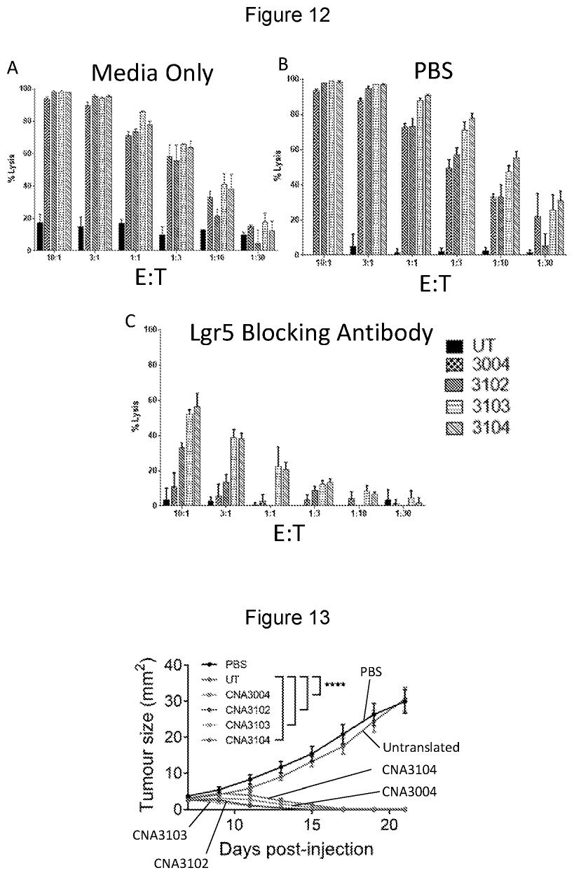

20 As can be seen in FIGS. 12A and 12B, cells which were not incubated with

antibodies (i.e. incubated with either XVIVO media only or PBS) were effectively and extensively killed by the CNA family CAR T cells (namely, CNA3004, CNA3102, CNA3103 and CNA3104) even at effector to target ratios as low as 1:10 and 1:30.

By comparison, FIG. 12C demonstrates that when target LoVo cells were 25 pre-incubated and co-incubated with the anti-Lgr5 antibody BNC101 the lysis of the target cells by the tested CNA family CAR T-cells was considerably reduced compared to the control XVIVO and PBS incubated target cells.

Example 8 — CNA CAR T-cells effectively reduce tumour growth in vivo

To assess if the CNA family CAR T-cells were effective at preventing or reducing cancer cell growth in vivo a murine xenograft cancer model was utilised as described below.

Animals

Male immunocompromised NOD.Cg-Prkdcsc'd 112rgtmlwil/SzJ (NSG) mice

between five and six weeks old were purchased from the Animal Resource Centre (Perth, WA). Mice were housed in pathogen-free conditions with a 12-hour light/dark cycle and were allowed to acclimatise for at least one week. All experiments were conducted under ethics approval.

Tissue Culture

The human colorectal adenocarcinoma LoVo cell line was grown and maintained in RPMI (Gibco) supplemented with 10% heat-inactivated fetal calf serum (FCS; Corning) and 100U/m1 penicillin/streptomycin (Life Technologies) and were cultured at 37° C. in 5% CO2. Cells were passaged every 2-3 days by rinsing the flasks with sterile PBS and dissociating cells with trypsin/EDTA in PBS (Gibco) for approximately 4 min at 37° C.

CAR-T Manufacture

CD3+Lgr5-targeting CAR-T cells were generated as described above. Specifically, CNA3004 (light chain-heavy chain orientation with a long linker), CNA3102 (heavy chain-light chain orientation with a short linker), CNA3103 (heavy chain-light chain orientation with a medium linker) and CNA3104 (heavy chain-light chain orientation with a long linker) were tested in this model.

Xenograft Mouse Model

Six to seven-week-old NSG mice were injected subcutaneously into the lower abdomen with 2x10 6 LoVo human colorectal adenocarcinoma cells resuspended in sterile PBS. Each group consisted of between 5 and 7 mice.

A total of 2x10 7 live anti-Lgr5 CAR T-cells (specifically CNA3004, CNA3102, CNA3103 and CNA3104) in sterile Dulbecco's PBS were injected intravenously into mice three days after injection of cancer cells. Untransduced T-cells (2x10 7) were administered to mice as a negative control with a second control group consisting of mice administered with PBS alone.

Tumour size was measured every 2 days beginning on day 7 using digital callipers by measuring the longest distance as length and the perpendicular distance as width. Tumour area was calculated as length x width. In vivo procedures including injections, tumour measurement and monitoring were conducted as a blinded experiment.

The health status of mice was monitored daily and mice were euthanized by CO2 when the tumours became ulcerated, tumour length was equal to or greater than 15mm, tumour area was equal or greater than 100mm 2 or when mice displayed a combination of disease symptoms including any of the following: ruffled coat, hunched posture, reluctance to move, laboured breathing, weight loss of 10% or more of initial weight and/or changes in behaviour or gait. Further, survival and days free of tumour were monitored in mice up to 100 days.

Statistical analysis

All statistical analyses were performed using GraphPad Prism. Statistics were performed using a Two-way ANOVA test with Bonferroni's post-test. **** =p<1.0001. or Log-rank analysis (Mantel-Cox) with significance values as indicated in the figure.

As can be seen in FIGS. 13A all four CNA CAR T-cells statistically

significantly reduced tumour size by day 21 with no or little detectable tumour at day 21 in mice administered with the CNA CAR T-cells. Accordingly, the CNA CAR T-cells function in vivo to prevent or reduce cancer tumour formation in a mouse cancer model indicating functionality in vivo.

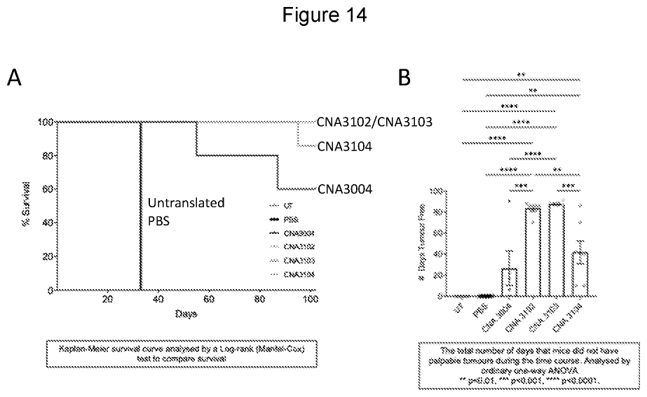

As can be seen in FIG. 14A and 14B, while all four CNA CAR T-cells

statistically significantly reduced tumour size by day 21 with no or little detectable tumour at day 21 in mice administered with the CNA CAR T-cells, CNA3102 and CNA3103 were superior to CNA3104 and CNA3004 in terms of both mouse survival out to day 100 (FIG. 14A) and days tumour-free (FIG. 14B).

1. A chimeric antigen receptor including:
an extracellular domain including a binding domain which recognises Lgr5;
a transmembrane domain; and
an intracellular signalling domain that activates a cellular function.

2-3. (canceled)

4. The chimeric antigen receptor of claim 1, wherein
the binding domain includes a variable heavy chain of an antibody that binds to Lgr5, or a functional variant of the variable heavy chain.
or includes a variable light chain of the antibody that binds to Lgr5, or a functional variant of the variable light chain.

5-6. (canceled)

7. The chimeric antigen receptor of claim 1, wherein the binding domain includes:
a heavy chain CDR1 having an amino acid sequence as set forth in SEQ ID No. 37 or having an amino acid sequence as set forth in SEQ ID No. 37 with up to 2 amino acid modifications,
a heavy chain CDR2 having an amino acid sequence as set forth in SEQ ID No. 38, or having an amino acid sequence as set forth in SEQ ID No. 38 with up to 2 amino acid modifications, and
a heavy chain CDR3 having an amino acid sequence as set forth in SEQ ID No. 39, or having an amino acid sequence as set forth in SEQ ID No. 39 with up to 2 amino acid modifications; and/or
a light chain CDR1 having an amino acid sequence set forth in SEQ ID No. 40 or having an amino acid sequence as set forth in SEQ ID No. 40 with up to 2 amino acid modifications,
a light chain CDR2 having an amino acid sequence set forth in SEQ ID No. 41 or having an amino acid sequence as set forth in SEQ ID No. 41 with up to 2 amino acid modifications. and
a light chain CDR3 having an amino acid sequence set forth in SEQ ID No. 42 or having an amino acid sequence as set forth in SEQ ID No. 42 with up to 2 amino acid modifications.

8-9. (canceled)

10. The chimeric antigen receptor of claim 1,
wherein the binding domain includes C-terminus of a variable heavy (VH) chain linked to N-terminus of a variable light (VL) chain,
or wherein a single-chain variable fragment includes the C-terminus of the variable light (VL) chain linked to the N-terminus of the variable light (VH) chain.

11. (canceled)

12. The chimeric antigen receptor of claim 1,
wherein the binding domain includes SEQ ID No. 49 and/or SEQ ID No. 50, or variants thereof having at least 90% sequence identity to SEQ ID No. 49 or 50,
or wherein the binding domain includes SEQ ID No. 53 or SEW ID No. 54,or variants thereof having at least 90% sequence identity to SEQ No. 53or 54.

13. (canceled)

14. The chimeric antigen receptor of claim 1, wherein the extracellular domain includes a linker domain which links the binding domain to the transmembrane domain.

15. The chimeric antigen receptor of claim 14, wherein the linker domain is at least 12 amino acids in length, or is at least about 12 amino acids in length, or is at least 119 amino acids in length, or is at least about 119 amino acids in length, or is at least 229 amino acids in length, or is at least about 229 amino acids in length.

16-17. (canceled)

18. The chimeric antigen receptor of claim 15, wherein the linker domain is up to 119 amino acids in length, or is up to about 119 amino acids in length, or is up to 229 amino acids in length, or up to about 229 amino acids in length.

19-20. (canceled)

21. The chimeric antigen receptor of claim 14, wherein the linker domain includes, or is selected from a croup consisting of, SEQ ID No. 55, SEQ ID No. 56, SEQ ID No. 57, or functional variants thereof.

22-29. (canceled)

30. The chimeric antigen receptor of claim 1, wherein the intracellular signalling domain includes SEQ ID No. 58 and/or SEQ ID No. 59.

31. A chimeric antigen receptor comprising, or consisting of, an amino acid sequence selected from the group consisting of: SEQ ID No. 60, 61, 62, 63, 64 and 65, and functional variants thereof.

32. (canceled)

33. A nucleic acid molecule including a nucleotide sequence encoding the chimeric antigen receptor of claim 1.

34-39. (canceled)

40. A viral vector including the nucleic acid molecule of claim 33.

41-42. (canceled)

43. A method of preparing a genetically modified cell, the method comprising using the viral vector of claim 40 to modify a cell.

44-46. (canceled)

47. A genetically modified cell, including the nucleic acid molecule of claim 33, or a genomically integrated form of the nucleic acid molecule.

48. The genetically modified cell of claim 47, wherein the cell is a leukocyte, a peripheral blood mononuclear cell (PBMC), a lymphocyte, a T cell, a natural killer cell, or a natural killer T cell.

49-50. (canceled)

51. A method of killing a target cell expressing or aberrantly-expressing Lgr5, the method including exposing the target cell to a genetically modified cell including the chimeric antigen receptor according to claim 1.

52-56 (canceled)

57. The method of claim 51, wherein the target cell is a cancer cell of one or more of breast cancer, pancreatic cancer, prostate cancer cancer, colon cancer, colorectal cancer, gastrointestinal cancer, lung cancer, lymphoma, ovarian cancer, B-cell lymphoma, neuroblastoma, glioblastoma brain cancer, oesophageal cancer, thyroid cancer, mouth cancer, head and neck cancer, or tongue cancer.

58-61. (canceled)

62. A method of preventing or treating a cancer associated with Lgr5 expression, the method comprising administering to a patient the chimeric antigen receptor according to claim 1.

63-68. (canceled)

69. A pharmaceutical composition including a nucleic acid molecule according to claim 33 or a genetically modified cell including the nucleic acid molecule, and further including a pharmaceutically acceptable carrier, excipient or diluent.

70. (canceled)
